(19) United States
(12) Patent Application Publication
(10) Pub. No.: US 2006/0105948 A1
(43) Pub. Date: May 18, 2006

(54) GLP-2 derivatives

(75) Inventors: Janos Tibor Kodra, Copenhagen (DK); Nils Langeland Johansen, Copenhagen (DK); Lars Thim, Gentofte (DK); Bernd Peschke, Malov (DK)

Correspondence Address: NOVO NORDISK, INC.;PATENT DEPARTMENT, 100 COLLEGE ROAD WEST, PRINCETON, NJ, 08540, US

(73) Assignee: Novo Nordisk A/S, Bagsvaerd (DK)

(21) Appl. No.: 11/235,737

(22) Filed: Sep. 22, 2005

Related U.S. Application Data
(63) Continuation of application No. PCT/DK04/00198, filed on Mar. 23, 2004.
(60) Provisional application No. 60/459,838, filed on Apr. 2, 2003.

(30) Foreign Application Priority Data
Mar. 24, 2003 (DK) PA 2003 00451

(51) Int. Cl. C07K 14/605; A61K 38/26

(52) U.S. Cl. 514/12; 530/324

(57) ABSTRACT

The present invention relates to novel derivatives of human glucagon-like peptide-2 (GLP-2) peptides, which have a protracted profile of action, as well as pharmaceutical compositions, uses and methods of treatment.

### CROSS-REFERENCE TO RELATED PATENT APPLICATIONS

This patent application is a continuation of PCT/DK2004/000198 (published as WO 2004/085471), filed Mar. 23, 2004 (and designating the United States), and claims priority to U.S. Provisional Patent Application 60/459,838, filed Apr. 2, 2003, and Danish Patent Application PA 2003 00451, filed Mar. 24, 2003, the contents of all of which are hereby incorporated by reference.

### FIELD OF THE INVENTION

The present invention relates to novel human glucagon-like peptide 2 (GLP-2) peptides and derivatives thereof which have a protracted profile of action. The invention further relates to methods of making and using these GLP-2 peptides and derivatives as well as polynucleotide constructs encoding such GLP-2 peptides and host cells comprising and expressing the GLP-2 peptides, pharmaceutical compositions, uses and methods of treatment.

### BACKGROUND OF THE INVENTION

Glucagon-like peptide 2 (GLP-2) is a 33 amino acid residue peptide produced in intestinal L-cells and released following nutrient intake. The amino acid sequence of the human GLP-2 peptide is given in FIG. 1.

The GLP-2 peptide is a product of the proglucagon gene. Proglucagon is expressed mainly in the pancreas and the intestine and to some extent in specific neurons located in the brain. The posttranslational processing of proglucagon is however different in pancreas and intestine. In the pancreas proglucagon is processed mainly to Glucagon Related Pancreatic Polypeptide (GRPP), Glucagon and Major Proglucagon Fragment. In contrast to this the processing in the intestine results in Glicentin, Glucagon-Like Peptide 1 (GLP-1) and Glucagon-Like Peptide 2 (GLP-2).

GLP-2 is secreted from the L-cells in the small and large intestine. This secretion is regulated by nutrient intake. The plasma concentration of GLP-2 in normal fasting subjects is around 15 pM increasing to around 60 pM after a mixed meal.

The actions of GLP-2 are transduced by a recently cloned glucagon-like peptide-2 receptor. The GLP-2 receptor represents a new member of the G protein-coupled 7TM receptor superfamily. The GLP-2R is expressed in a highly tissue-specific manner predominantly in the gastrointestinal tract and GLP-2R activation is coupled to increased adenylate cyclase activity. Cells expressing the GLP-2R responds to GLP-2, but not to other peptide of the glucagon family (Glucagon, GLP-1 and GIP).

In the rat the GLP-2R has also been reported to be expressed in the brain or more specific the dorsomedial hypothalamic nucleus. This part of the brain is normally thought to be involved in feeding behaviour and it has been shown that GLP-2 inhibits food intake when injected directly into the brain.

Induction of intestinal epithelial proliferation by GLP-2 was demonstrated (Drucker, D. J. et al (1996) Proc. Natl. Acad. Sci. USA 93: 7911-7916) and treatment of gastrointestinal deseases by cells grown in medium containing GLP-2 was disclosed (Drucker, D. J and Keneford, J. R., WO 96/32414).

WO 97/31943 relates to GLP-2 peptide analogs and the use of certain GLP-2 peptide analogs for appetite suppression or satiety induction.

WO 98/08872 Relates to GLP-2 derivatives comprising a lipophilic substituent.

WO 96/32414 and WO 97/39031 relates to specific GLP-2 peptide analogs.

WO 98/03547 relates to specific GLP-2 peptide analogs, which exhibit antagonist activity

GLP-2 peptides and derivatives thereof are useful in the treatment of gastrointestinal disorders. One problem associated with the use of GLP-2 relates to its short biological half-life (about 7 min). The GLP-2 is subject to enzymatic degradation and is rapidly degraded in the plasma via dipeptidylpeptidase IV (DPP-IV) cleavage between residues Ala² and Asp³

Accordingly, it is an object of the present invention to provide derivatives of GLP-2 which have a protracted profile of action relative to native GLP-2, while still retaining the GLP-2 activity. It is a further object of the invention to provide a pharmaceutical composition comprising a compound according to the invention and to use a compound of the invention to provide such a composition. Also, it is an object of the present invention to provide a method of treating gastrointestinal disorders.

### SUMMARY OF THE INVENTION

Described are new GLP-2 derivatives to be used for the continuous presence of a therapeutically effective amount of a compound acting via the GLP-2 mediated pathway. The protracted profile effect of these new GLP-2 derivatives is achieved by coupling of a GLP-2 peptide to a hydrophilic moiety that results in GLP-2 derivatives with an improved half-life, thereby facilitating the continuous presence of therapeutically effective amount of GLP-2. Among the preferred hydrophilic moieties that result in continuous presence of GLP-2 are covalently attached hydrophilic polymers such as polyethylene glycol and polypropylene glycol that reduce clearance and are not immunogenic. Disclosed are novel modified forms of GLP-2 derivates having specific amino acid residues modified by covalently attaching polyethyleneglycol (PEG).

Polyethylene glycol (PEG) is a hydrophilic, biocompatible and non-toxic polymer of general formula H(OCH₂CH₂)ₙOH wherein n>4. Its molecular weight could vary from 200 to 100.000 Daltons.

The in vivo half-life of certain therapeutic proteins and peptides has been increased by conjugating the protein or peptide with PEG, which is termed “pegylation”. See, e.g. Abuchowski et al., J. Biol. Chem., 252: 3582-3586 (1977), PEG provides a protective coating and increases the size of the molecule, thus reducing its metabolic degredation and its renal clearance. In addition, pegylation has been reported to reduce immunogenicity and toxicity of certain therapeutic proteins. Abuchowski et al. J. Biol. Chem., 252: 3578-3581 (1977).

In its broadest aspect, the present invention relates to derivatives of GLP-2 peptides. The derivatives according to the invention have interesting pharmacological properties, in particular they have a more protracted profile of action than the parent GLP-2 peptides.

In a first aspect, the invention relates to a GLP-2 derivative comprising a GLP-2 peptide, wherein a hydrophilic substituent is attached to one or more amino acid residues at a position relative to the amino acid sequence of SEQ ID NO:1.

In a second aspect, the invention relates to a GLP-2 derivative comprising a GLP-2 peptide, wherein a hydrophilic substituent is attached to one or more amino acid residues at a position relative to the amino acid sequence of SEQ ID NO:1 independently selected from the list consisting of D3, S5, S7, D8, E9, M10, N11, T12, I13, L14, D15, N16, L17, A18, R20, D21, N24, Q28, and D33.

In a third aspect, the invention relates to a pharmaceutical composition comprising a GLP-2 derivative comprising a GLP-2 peptide, wherein a hydrophilic substituent is attached to one or more amino acid residues at a position relative to the amino acid sequence of SEQ ID NO: 1.

In a further aspect, the invention relates to a pharmaceutical composition comprising a GLP-2 derivative comprising a GLP-2 peptide, wherein a hydrophilic substituent is attached to one or more amino acid residues at a position relative to the amino acid sequence of SEQ ID NO:1 independently selected from the list consisting of D3, S5, S7, D8, E9, M10, N11, T12, I13, L14, D15, N16, L17, A18, R20, D21, N24, Q28, and D33.

In one embodiment a hydrophilic substituent is attached to an amino acid residue at the position D3 relative to the amino acid sequence of SEQ ID NO:1. In one embodiment a hydrophilic substituent is attached to an amino acid residue at the position S5 relative to the amino acid sequence of SEQ ID NO:1. In one embodiment a hydrophilic substituent is attached to an amino acid residue at the position S7 relative to the amino acid sequence of SEQ ID NO:1. In one embodiment a hydrophilic substituent is attached to an amino acid residue at the position D8 relative to the amino acid sequence of SEQ ID NO: 1. In one embodiment a hydrophilic substituent is attached to an amino acid residue at the position E9 relative to the amino acid sequence of SEQ ID NO:1. In one embodiment a hydrophilic substituent is attached to an amino acid residue at the position M10 relative to the amino acid sequence of SEQ ID NO:1. In one embodiment a hydrophilic substituent is attached to an amino acid residue at the position N11 relative to the amino acid sequence of SEQ ID NO:1. In one embodiment a hydrophilic substituent is attached to an amino acid residue at the position T12 relative to the amino acid sequence of SEQ ID NO:1. In one embodiment a hydrophilic substituent is attached to an amino acid residue at the position I13 relative to the amino acid sequence of SEQ ID NO:1. In one embodiment a hydrophilic substituent is attached to an amino acid residue at the position L14 relative to the amino acid sequence of SEQ ID NO:1. In one embodiment a hydrophilic substituent is attached to an amino acid residue at the position D15 relative to the amino acid sequence of SEQ ID NO:1. In one embodiment a hydrophilic substituent is attached to an amino acid residue at the position N16 relative to the amino acid sequence of SEQ ID NO:1. In one embodiment a hydrophilic substituent is attached to an amino acid residue at the position L17 relative to the amino acid sequence of SEQ ID NO:1. In one embodiment a hydrophilic substituent is attached to an amino acid residue at the position A18 relative to the amino acid sequence of SEQ ID NO:1. In one embodiment a hydrophilic substituent is attached to an amino acid residue at the position R20 relative to the amino acid sequence of SEQ ID NO:1. In one embodiment a hydrophilic substituent is attached to an amino acid residue at the position D21 relative to the amino acid sequence of SEQ ID NO:1. In one embodiment a hydrophilic substituent is attached to an amino acid residue at the position N24 relative to the amino acid sequence of SEQ ID NO:1. In one embodiment a hydrophilic substituent is attached to an amino acid residue at the position Q28 relative to the amino acid sequence of SEQ ID NO:1. In one embodiment a hydrophilic substituent is attached to an amino acid residue at the position D33 relative to the amino acid sequence of SEQ ID NO:1. It is to be understood that an amino acid residues at the position relative to the amino acid sequence of SEQ ID NO:1 may be any amino acid residue and not only the amino acid residue naturally present at that position. In one embodiment the hydrophilic substituent is attached to a lysine.

In a further aspect, the invention relates to the use of a GLP-2 derivative comprising a GLP-2 peptide, wherein a hydrophilic substituent is attached to one or more amino acid residues at a position relative to the amino acid sequence of SEQ ID NO:1 for the preparation of a medicament.

In a further aspect, the invention relates to the use of a GLP-2 derivative comprising a GLP-2 peptide, wherein a hydrophilic substituent is attached to one or more amino acid residues at a position relative to the amino acid sequence of SEQ ID NO:1 independently selected from the list consisting of D3, S5, S7, D8, E9, M10, N11, T12, I13, L14, D15, N16, N17, A18, R20, D21, N24, Q28, and D33 for the preparation of a medicament.

In a further aspect, the invention relates to the use of a GLP-2 derivative comprising a GLP-2 peptide, wherein a hydrophilic substituent is attached to one or more amino acid residues at a position relative to the amino acid sequence of SEQ ID NO:1 for the preparation of a medicament with protracted effect.

In a further aspect, the invention relates to the use of a GLP-2 derivative comprising a GLP-2 peptide, wherein a hydrophilic-substituent is attached to one or more amino acid residues at a position relative to the amino acid sequence of SEQ ID NO:1 independently selected from the list consisting of D3, S5, S7, D8, E9, M10, N11, T12, I13, L14, D15, N16, D21, N24, Q28, and D33 for the preparation of a medicament with protracted effect.

In a further aspect, the invention relates to the use of a GLP-2 derivative comprising a GLP-2 peptide, wherein a hydrophilic substituent is attached to one or more amino acid residues at a position relative to the amino acid sequence of SEQ ID NO:1 for the preparation of a medicament for the treatment of intestinal failure or other condition leading to malabsorption of nutrients in the intestine.

In a further aspect, the invention relates to the use of a GLP-2 derivative comprising a GLP-2 peptide, wherein a hydrophilic substituent is attached to one or more amino acid residues at a position relative to the amino acid sequence of SEQ ID NO:1 independently selected from the list consisting of D3, S5, S7, D8, E9, M10, N11, T12, I13, L14, D15, N16, L17, A18, R20, D21, N24, Q28, and D33 for the preparation of a medicament for the treatment of intestinal failure or other condition leading to malabsorption of nutrients in the intestine.

In a further aspect, the invention relates to the use of a GLP-2 derivative comprising a GLP-2 peptide, wherein a hydrophilic substituent is attached to one or more amino acid residues at a position relative to the amino acid sequence of SEQ ID NO:1 for the preparation of a medicament for the treatment of small bowel syndrome, Inflammatory bowel syndrome, Crohns disease, colitis including collagen colitis, radiation colitis, post radiation atrophy, non-tropical (gluten intolerance) and tropical sprue, damaged tissue after vascular obstruction or trauma, tourist diarrhea, dehydration, bacteremia, sepsis, anorexia nervosa, damaged tissue after chemotherapy, premature infants, schleroderma, gastritis including atrophic gastritis, postantrectomy atrophic gastritis and helicobacter pylori gastritis, ulcers, enteritis, cul-de-sac, lymphatic obstruction, vascular disease and graft-versus-host, healing after surgical procedures, post radiation atrophy and chemotherapy, and osteoporosis.

In a further aspect, the invention relates to the use of a GLP-2 derivative comprising a GLP-2 peptide, wherein a hydrophilic substituent is attached to one or more amino acid residues at a position relative to the amino acid sequence of SEQ ID NO:1 independently selected from the list consisting of D3, S5, S7, D8, E9, M10, N11, T12, I13, L14, D15, N16, L17, A18, R20, D21, N24, Q28, and D33 for the preparation of a medicament for the treatment of small bowel syndrome, Inflammatory bowel syndrome, Crohns disease, colitis including collagen colitis, radiation colitis, post radiation atrophy, non-tropical (gluten intolerance) and tropical sprue, damaged tissue after vascular obstruction or trauma, tourist diarrhea, dehydration, bacteremia, sepsis, anorexia nervosa, damaged tissue after chemotherapy, premature infants, schleroderma, gastritis including atrophic gastritis, postantrectomy atrophic gastritis and helicobacter pylori gastritis, ulcers, enteritis, cul-de-sac, lymphatic obstruction, vascular disease and graft-versus-host, healing after surgical procedures, post radiation atrophy and chemotherapy, and osteoporosis.

In a further aspect, the invention relates a method for the treatment of instestinal failure or other condition leading to malabsorption of nutrients in the intestine, the method comprising administering a therapeutically or prophylactically effective amount of a GLP-2 derivative comprising a GLP-2 peptide, wherein a hydrophilic substituent is attached to one or more amino acid residues at a position relative to the amino acid sequence of SEQ ID NO:1; to a subject in need thereof.

In a further aspect, the invention relates a method for the treatment of instestinal failure or other condition leading to malabsorption of nutrients in the intestine, the method comprising administering a therapeutically or prophylactically effective amount of a GLP-2 derivative comprising a GLP-2 peptide, wherein a hydrophilic substituent is attached to one or more amino acid residues at a position relative to the amino acid sequence of SEQ ID NO:1 independently selected from the list consisting of D3, S5, S7, D8, E9, M10, N11, T12, I13, L14, D15, N16, L17, A18, R20, D21, N24, Q28, and D33; to a subject in need thereof.

In a further aspect, the invention relates a method for the treatment of small bowel syndrome, Inflammatory bowel syndrome, Crohns disease, colitis including collagen colitis, radiation colitis, post radiation atrophy, non-tropical (gluten intolerance) and tropical sprue, damaged tissue after vascular obstruction or trauma, tourist diarrhea, dehydration, bacteremia, sepsis, anorexia nervosa, damaged tissue after chemotherapy, premature infants, schleroderma, gastritis including atrophic gastritis, postantrectomy atrophic gastritis and helicobacter pylori gastritis, ulcers, enteritis, cul-de-sac, lymphatic obstruction, vascular disease and graft-versus-host, healing after surgical procedures, post radiation atrophy and chemotherapy, and osteoporosis, the method comprising administering a therapeutically or prophylactically effective amount of a GLP-2 derivative comprising a GLP-2 peptide, wherein a hydrophilic substituent is attached to one or more amino acid residues at a position relative to the amino acid sequence of SEQ ID NO:1; to a subject in need thereof.

In a further aspect, the invention relates a method for the treatment of small bowel syndrome, Inflammatory bowel syndrome, Crohns disease, colitis including collagen colitis, radiation colitis, post radiation atrophy, non-tropical (gluten intolerance) and tropical sprue, damaged tissue after vascular obstruction or trauma, tourist diarrhea, dehydration, bacteremia, sepsis, anorexia nervosa, damaged tissue after chemotherapy, premature infants, schleroderma, gastritis including atrophic gastritis, postantrectomy atrophic gastritis and helicobacter pylori gastritis, ulcers, enteritis, cul-de-sac, lymphatic obstruction, vascular disease and graft-versus-host, healing after surgical procedures, post radiation atrophy and chemotherapy, and osteoporosis, the method comprising administering a therapeutically or prophylactically effective amount of a GLP-2 derivative comprising a GLP-2 peptide, wherein a hydrophilic substituent is attached to one or more amino acid residues at a position relative to the amino acid sequence of SEQ ID NO:1 independently selected from the list consisting of D3, S5, S7, D8, E9, M10, N11, T12, I13, L14, D15, N16, L17, A18, R20, D21, subject in need thereof.

### DETAILED DESCRIPTION OF THE INVENTION

Short Bowel Syndrome (SBS) is a devastating clinical condition encountered in a wide spectrum of medical and surgical conditions. The most common causes include irradiation, cancer, mesenteric vascular disease, Crohn's disease and trauma. With improved care of patients with SBS a greater number of patients are surviving for a longer period of time, thus magnifying the need for thera-peutic interventions to reduce or eliminate the long-term problems associated with SBS. Although SBS patient have up to 7 meals a day they still have problems maintaining normal body weight and these patients are often maintained on parenteral nutrition either at home (HPN) or at the hospital.

Chemotherapy (CT) and radiation therapy (RT) for treatment of cancers target rapidly dividing cells. Since the cells of intestinal crypts (the simple tubular glands of the small intestine) are rapidly proliferating, CT/RT tends to produce intestinal mucosal damage as an adverse effect. Gastroenteritis, diarrhea, dehydration and, in some cases, bacteremia and sepsis may ensue. These side effects are severe for two reasons: They set the limit for the dose of therapy and thereby the efficacy of the treatment, and they represent a potentially life-threatening condition, which requires intensive and expensive treatment.

Animal studies have shown that CT-induced intestinal mucosal damage can be counteracted by GLP-2 peptides due to its potent intestinotrophic activity, which leads to an increase in bowel weight, villus height, crypt depth and crypt cell proliferation rate and, importantly, a reduction of crypt cell apoptosis. RT-induced GI tract damage and the potential protective effect of GLP-2 peptides follow the same rationale as that of CT.

Inflammatory bowel disease (IBD) comprises Crohn's disease, which mainly affects the small intestine, and ulcerative colitis, which mainly occurs in the distal colon and rectum. The pathology of IBD is characterized by chronic inflammation and destruction of the GI epithelium. Current treatment is directed towards suppression of inflammatory mediators. Stimulation of repair and regeneration of the epithelium by intestinotrophic agents such as GLP-2 derivatives according to the present invention might represent an alternative or adjunct strategy for treatment of IBD.

Dextran sulfate (DS)-induced colitis in rodents resembles ulcerative colitis in man, with development of mucosal edema, crypt erosions and abscesses, leading to polyp formation and progression to dysplasia and adenocarcinoma, but the precise mechanism underlying the toxicity of DS is not known. A beneficial effect of GLP-2 peptides in (DS)-induced colitis in mice have been demonstrated, Drucker et al. Am. J. Physiol. 276 (Gastrointest. Liver Physiol. 39): G79-G91, 1999. Mice receiving 5% DS in the drinking water developed loose blood-streaked stools after 4-5 days and lost 20-25% of their body weight after 9-10 days. Mice that were in addition treated subcutaneously twice daily for the whole period (9-10 days) with either 350 ng or 750 ng A2G-GLP-2(1-33) lost significantly less body weight and appeared much healthier. The effects were dose-dependent. By histology, DS mice treated with A2G-GLP-2(1-33) exhibited a greater proportion of intact mucosal epithelium, increased colon length, crypt depth and mucosal area. These effects were mediated in part via enhanced stimulation of mucosal epithelial cell proliferation. It is concluded by the inventors of the present invention that there is a therapeutic potential for the treatment of IBD of GLP-2 derivatives according to the present invention, potentially in combination with anti-inflammatory drugs. Thus, there is a potential of GLP-2 derivatives according to the present invention as an adjunct to anti-inflammatory therapy in IBD. The predominant role of GLP-2 derivatives according to the present invention in IBD would be to enhance the regeneration of compromised intestinal epithelium.

The degradation of native GLP-2(1-33) in vivo in humans presumably by Dipeptidyl Peptidase IV (DPP-IV) has been studied in details by Hartmann et al. (J Clin. Endocrinol Metab 85: 2884-2888, 2000). GLP-2 infusions (0.8 pmol/kg * min) increasing plasma level of intact GLP-2(1-33) from 9 pM to 131 pM was eliminated with T½ value of 7 min. When an s.c. injection of GLP-2(1-33) was given (400 mg =106.000 pmol) the plasma concentration increased to a maximum of 1500 pM after 45 min. One hour after the s.c. injection, 69% of the injected GLP-2(1-33) was still intact GLP-2(1-33). In both studies the only degradation product detected by HPLC was GLP-2(3-33) and it was concluded that GLP-2 is extensively degraded to GLP-2 (3-33) in humans presumably by DPP-IV. Thus the object of the present invention is to provide GLP-2 derivatives, that are resistant to DPP-IV degradation are thus more potent in vivo that the native GLP-2 peptide.

The term “GLP-2 peptide” as used herein means any protein comprising the amino acid sequence 1-33 of native human GLP-2 (SEQ ID NO: 1) or analogs thereof. This includes but are not limited to native human GLP-2 and analogs thereof.

The term “GLP-2” as used herein is intended to include proteins that have the amino acid sequence 1-33 of native human GLP-2 with amino acid sequence of SEQ ID NO:1. It also includes proteins with a slightly modified amino acid sequence, for instance, a modified N-terminal end including N-terminal amino acid deletions or additions so long as those proteins substantially retain the activity of GLP-2. “GLP-2” within the above definition also includes natural allelic variations that may exist and occur from one individual to another. Also, degree and location of glycosylation or other post-translation modifications may vary depending on the chosen host cells and the nature of the host cellular environment.

The terms “analog” or “analogs”, as used herein, is intended to designate a GLP-2 peptide having the sequence of SEQ ID NO: 1, wherein one or more amino acids of the parent GLP-2 protein have been substituted by another amino acid and/or wherein one or more amino acids of the parent GLP-2 protein have been deleted and/or wherein one or more amino acids have been inserted in protein and/or wherein one or more amino acids have been added to the parent GLP-2 protein. Such addition can take place either at the N-terminal end or at the C-terminal end of the parent GLP-2 protein or both. The “analog” or “analogs” within this definition still have GLP-2 activity as measured by the ability to exert a trophic effects on the small or large intestine. In one embodiment an analog is 70% identical with the sequence of SEQ ID NO:1. In one embodiment an analog is 80% identical with the sequence of SEQ ID NO:1. In another embodiment an analog is 90% identical with the sequence of SEQ ID NO:1. In a further embodiment an analog is 95% identical with the sequence of SEQ ID NO:1. In a further embodiment an analog is a GLP-2 peptide, wherein a total of up to ten amino acid residues of SEQ ID NO:1 have been exchanged with any amino acid residue. In a further embodiment an analog is a GLP-2 peptide, wherein a total of up to five amino acid residues of SEQ ID NO:1 have been exchanged with any amino acid residue. In a further embodiment an analog is a GLP-2 peptide, wherein a total of up to three amino acid residues of SEQ ID NO:1 have been exchanged with any amino acid residue. In a further embodiment an analog is a GLP-2 peptide, wherein a total of up to two amino acid residues of SEQ ID NO:1 have been exchanged with any amino acid residue. In a further embodiment an analog is a GLP-2 peptide, wherein a total of one amino acid residue of SEQ ID NO:1 have been exchanged with any amino acid residue.

The term “a fragment thereof”, as used herein, means any fragment of the peptide according to formula I or II with at least 15 amino acids. In one embodiment the fragment has at least 20 amino acids. In one embodiment the fragment has at least 25 amino acids. In one embodiment the fragment has at least 30 amino acids. In one embodiment the fragment is according to formula I or II with one amino acid deletion in the C-terminal. In one embodiment the fragment is according to formula I or II with two amino acid deletions in the C-terminal. In one embodiment the fragment is according to formula I or II with three amino acid deletions in the C-terminal. In one embodiment the fragment is according to formula I or II with four amino acid deletions in the C-terminal.

In one embodiment the fragment is according to formula I or II with one amino acid deletion in the N-terminal. In one embodiment the fragment is according to formula I or II with two amino acid deletions in the N-terminal. In one embodiment the fragment is according to formula I or II with three amino acid deletions in the N-terminal. In one embodiment the fragment is according to formula I or II with four amino acid deletions in the N-terminal.

The term “derivative” is used in the present text to designate a peptide in which one or more of the amino acid residues have been chemically modified, e.g. by alkylation, acylation, ester formation or amide formation.

The term “a GLP derivative” is used in the present text to designate a derivative of a GLP-2 peptide. In one embodiment the GLP-2 derivative according to the present invention has GLP-2 activity as measured by the ability to bind a GLP-2 receptor (GLP-2R) and/or exert a trophic effects on the small or large intestine. In one embodiment the GLP-2 receptor is selected from the list consisting of rat GLP-2R, mouse GLP-2R and human GLP-2R.

It is to be understood, that the hydrophilic substituent is attached to a GLP-2 peptide by covalent attachment. The term “covalent attachment” means that the GLP-2 peptide and the hydrophilic substituent is either directly covalently joined to one another, or else is indirectly covalently joined to one another through an intervening moiety or moieties, such as a bridge, spacer, or linkage moiety or moieties.

The term “hydrophilic substituent”, means a radical, which is formally derived from a hydrophilic, water soluble molecule by removal of a hydroxyl-radical, regardless of the actual synthesis chosen. The terms “hydrophilic” and “hydrophobic” are generally defined in terms of a partition coefficient P, which is the ratio of the equilibrium concentration of a compound in an organic phase to that in an aqueous phase. A hydrophilic compound has a log P value less than 1.0, typically less than about −0.5, where P is the partition coefficient of the compound between octanol and water, while hydrophobic compounds will generally have a log P greater than about 3.0, typically greater than about 5.0.

The polymer molecule is a molecule formed by covalent linkage of two or more monomers wherein none of the monomers is an amino acid residue. Preferred polymers are polymer molecules selected from the group consisting of polyalkylene oxides, including polyalkylene glycol (PAG), such as polyethylene glycol (PEG) and polypropylene glycol (PPG), branched PEGs, polyvinyl alcohol (PVA), polycarboxylate, poly-vinylpyrolidone, polyethylene-co-maleic acid anhydride, polystyrene-co-maleic acid anhydride, and dextran, including carboxymethyl-dextran, PEG being particular preferred. The term “attachment group” is intended to indicate a functional group of the GLP-2 or the GLP-2 derivative capable of attaching a polymer molecule. Useful attachment groups are, for example, amine, hydroxyl, carboxyl, aldehyde, ketone, sulfhydryl, succinimidyl, maleimide, vinylsulfone, oxime or halo acetate.

The term “PAO” as used herein refers to any polyalkylene oxide, including polyalkylene glycol (PAG), such as polyethylene glycol (PEG) and polypropylene glycol (PPG), branched PEGs and methoxypolyethylene glycol (mPEG) with a molecular weight from about 200 to about 100.000 Daltons. In one embodiment the PAO is a polyalkylene glycol (PAG). In one embodiment the PAO is a polyethylene glycol (PEG). In one embodiment the PAO is a polypropylene glycol (PPG). In one embodiment the PAO is a branched PEG. In one embodiment the PAO is a methoxypolyethylene glycol (mPEG).

When a polymer, such as those polymers described as PAO, PAG or PEG, is attached to GLP-2 or a GLP-2 derivative through a covalent bond, it is intended without being said expressis verbis, that the attachment can be direct without a further linker or with a linker.

Especially preferred are those compounds where the polymer is attached to GLP-2 or a GLP-2 derivative through no linker or where the linker is selected of the groups of C₃-₈-alkylene, carbonyl, C₃₋₈-alkyleneaminocarbonyl,

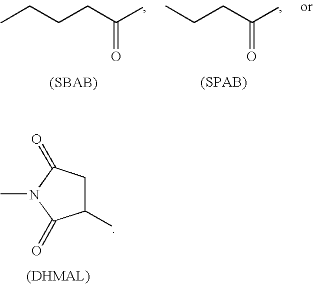

More preferred are those compounds where the linker is SBAB or SPAB.

In the present context, the term “treatment” is meant to include both prevention of an expected instestinal failure or other condition leading to malabsorption of nutrients in the intestine, such as in post radiation atrophy, and regulation of an already occurring instestinal failure, such as in Inflammatory bowel syndrome, with the purpose of inhibiting or minimising the effect of the condition leading to malabsorption of nutrients in the intestine. Prophylactic administration with the GLP-2 derivative according to the invention is thus included in the term “treatment”.

The term “subject” as used herein is intended to mean any animal, in particular mammals, such as humans, and may, where appropriate, be used interchangeably with the term “patient”.

To obtain a satisfactory protracted profile of action of the GLP-2 derivative, the macromolecule are covalently attached polymers such as polyethylene glycol or polypropylene glycol; and other hydrophilic macromolecules, e.g. polysaccharides such as dextran, that reduce clearance and are not immunogenic.

The polymer molecule to be coupled to the GLP-2 peptide may be any suitable molecule such as natural or synthetic homo-polymer or hetero-polymer, typically with a molecular weight in the range of about 300-100.000 Da, such as about 500-20.000 Da, or about 500-15.000 Da, or 2-15 kDa, or 3-15 kDa, or about 10 kDa.

When the term “about” is used herein in connection with a certain molecular weight the word “about” indicates an approximate average molecular weight distribution in a given polymer preparation.

Examples of homo-polymers include a polyalcohol (i.e., poly-OH), a polyamine (i.e., poly-NH₂) and a polycarboxylic acid (i.e., poly-COOH). A hetero-polymer is a polymer comprising different coupling groups such as hydroxyl group and amine group.

Examples of suitable polymer molecules include polymer molecule selected from the group consisting of polyalkylene oxide, including polyalkylene glycol (PAG), such as polyethylene glycol (PEG) and polypropylene glycol (PPG), branched PEGs, polyvinyl alcohol (PVA), polycarboxylate, poly-vinylpyrolidone, polyethylene-co-maleic acid anhydride, polystyrene-co-maleic acid anhydride, dextran, including carboxymethyl-dextran, or any other polymer suitable for reducing immunicenicity and/or increasing functional in vivo half-life and/or serum half-life. Generally, polyalkyleneglycol-derived polymers are biocompatible, non-toxic, non-antigenic, and non-immunogenic, have various water solubility properties, and are easily secreted from living organism.

PEG is the preferred polymer molecule, since it has only a few reactive groups capable of cross-linking compared to e.g. polysaccharides such as dextran. In particular, mono-functional PEG, e.g., methoxypolyethylene glycol (mPEG) is of interest since its coupling chemistry is relatively simple (only one reactive group is available for conjugating with attachment groups the peptide).

To effect covalent attachment of the polymer molecule(s) to a GLP-2 peptide, the hydroxyl end groups of the polymer molecule must be provided in activated form, i.e. with reactive functional groups (examples of which includes primary amino groups, hydrazide (HZ), thiol (SH), succinate (SUC), succinimidyl succinate (SS), succinimidyl succinamide (SSA), succinimidyl proprionate (SPA), succinimidyl 3-mercaptopropionate (SSPA), Norleucine (NOR), succinimidyl carboxymethylate (SCM), succimidyl butanoate (SBA), succinimidyl carbonate (SC), succinimidyl glutarate (SG), acetaldehyde diethyl acetal (ACET), succinimidy carboxymethylate (SCM), benzotriazole carbonate (BTC), N-hydroxysuccinimide (NHS), aldehyde (ALD), trichlorophenyl carbonate (TCP) nitrophenylcarbonate (NPC), maleimide (MAL) vinylsulfone (VS), carbonylimidazole (CDI), isocyanate (NCO), iodine (IODO), expoxide (EPOX), iodoacetamide (IA), succinimidyl glutarate (SG) and tresylate (TRES).

Suitable activated polymer molecules are commercially available, e.g. from Nektar, formerly known as Shearwater Polymers, Inc., Huntsville, Ala., USA, or from PolyMASC Pharmaceuticals plc, UK or from Enzon pharmaceuticals. Alternatively, the polymer molecules can be activated by conventional methods known in the art, e.g. as disclosed in WO 90/13540. Specific examples of activated linear or branched polymer molecules for use in the present invention are described in the Shearwater Polymers, Inc. 1997 and 2000 Catalogs (Functionalized Biocompatible Polymers for Research and pharmaceuticals, Polyethylene Glycol and Derivatives, incorporated herein by reference).

Specific examples of activated PEG polymers include the following linear PEGs: NHS-PEG (e.g. SPA-PEG, SSPA-PEG, SBA-PEG, SS-PEG, SSA-PEG, SC-PEG, SG-PEG, and SCM-PEG), and NOR-PEG, SCM-PEG, BTC-PEG, EPOX-PEG, NCO-PEG, NPC-PEG, CDI-PEG, ALD-PEG, TRES-PEG, VS-PEG, IODO-PEG, IA-PEG, ACET-PEG and MAL-PEG, and branched PEGs such as PEG2-NHS and those disclosed in U.S. Pat. No. 5,672,662, U.S. Pat. No. 5,932,462 and U.S. Pat. No. 5,643,575 both which are incorporated herein by reference. Furthermore the following publications, incorporated herein by reference, disclose useful polymer molecules and/or PEGylation chemistries: U.S. Pat. No. 4,179,337, U.S. Pat. No. 5,824,778, U.S. Pat. No. 5,476,653, WO 97/32607, EP 229,108, EP 402,378, U.S. Pat. No. 4,902,502, U.S. Pat. No. 5,281,698, U.S. Pat. No. 5,122,614, U.S. Pat. No. 5,219,564, WO 92/16555, WO 94/04193, WO 94/14758, US 94/17039, WO 94/18247, WO 94,28024, WO 95/00162, WO 95/11924, WO 95/13090, WO 95/33490, WO 96/00080, WO 97/18832, WO 98/41562, WO 98/48837, WO 99/32134, WO 99/32139, WO 99/32140, WO 96/40791, WO 98/32466, WO 95/06058, EP 439 508, WO 97/03106, WO 96/21469, WO 95/13312, EP 921 131, U.S. Pat. No. 5,736,625, WO 98/05363, EP 809 996, U.S. Pat. No. 5,629,384, WO 96/41813, WO 96/07670, U.S. Pat. No. 5,473,034, U.S. Pat. No. 5,516,673, EP 605 963, EP 510 356, EP 400 472, EP 183 503 and EP 154 316 and Roberts et al. Adv. Drug Delivery Rev. 54: 459-476 (2002) and references described herein. The conjugation between a GLP-2 peptide and the activated polymer is conducted by conventional method. Conventional methods are known to those skilled in the art.

It will be understood that the polymer conjugation is designed so as to produce the optimal molecule with respect to the number of polymer molecules attached, the size and form of such molecules (e.g. whether they are linear or branched), and the attachment site(s) on GLP-2 or GLP-2 derivate. The molecular weight of the polymer to be used may e.g., be chosen on the basis of the desired effect to be achieved.

The hydrophilic substituent may be attached to an amino group of the GLP-2 moiety by means of a carboxyl group of the hydrophilic substituent which forms an amide bond with an amino group of the amino acid to which it is attached. As an alternative, the hydrophilic substituent may be attached to said amino acid in such a way that an amino group of the hydrophilic substituent forms an amide bond with a carboxyl group of the amino acid. As a further option, the hydrophilic substituent may be linked to the GLP-2 moiety via an ester bond. Formally, the ester can be formed either by reaction between a carboxyl group of the GLP-2 moiety and a hydroxyl group of the substituent-to-be or by reaction between a hydroxyl group of the GLP-2 moiety and a carboxyl group of the substituent-to-be. As a further alternative, the hydrophilic substituent can be an alkyl group which is introduced into a primary amino group of the GLP-2 moiety.

In one embodiment of the invention the GLP-2 derivative comprises a GLP-2 peptide comprising the amino acid sequence of formula II
His-X²-X³-Gly-X⁵-Phe-X⁷-X⁸-X⁹-X¹⁰-X¹¹-X¹²-X¹³-X¹⁴-X¹⁵-X¹⁶-X¹⁷-X¹⁸-Ala-X²⁰-X²¹-Phe-Ile-X²⁴-Trp-Leu-Ile-X²⁸-Thr-X³⁰-Ile-Thr-X³³ (formula II)
or a fragment thereof; wherein X² is Ala, Val or Gly; X³ is Asp, or Glu; X⁵ is Ser, or Lys; X⁷ is Ser, or Lys; X⁸ is Asp, Glu, or Lys; X⁹ is Asp, Glu, or Lys; X¹⁰ is Met, Lys, Leu, Ile, or Nor-Leucine; X¹¹ is Asn, or Lys; X¹² is Thr, or Lys; X¹³ is Ile, or Lys; X¹⁴ is Leu, or Lys; X¹⁵ is Asp, or Lys; X¹⁶ is Asn, or Lys; X¹⁷ is Leu, or Lys; X¹⁸ is Ala, or Lys; X²⁰ is Arg, or Lys; X²¹ is Asp, or Lys; X²⁴ is Asn, or Lys; X²⁸ is Gln, or Lys; X³⁰ is Arg, or Lys; X³³ is Asp, Glu, Lys, Asp-Arg, or Asp-Lys (formula II).

In one embodiment of the invention the GLP-2 derivative comprises a GLP-2 peptide GLP-2 peptide comprising the amino acid sequence
His-X²-X³-Gly-X⁵-Phe-X⁷-X⁸-X⁹-X¹⁰-X¹¹-X¹²-X¹³-X¹⁴-X¹⁵-X¹⁶-X¹⁷-X¹⁸-Ala-X²⁰-X²¹-Phe-Ile-X²⁴-Trp-Leu-Ile-X²⁸-Thr-X³⁰-Ile-Thr-X³³

or a fragment thereof; wherein X² is Ala, Val or Gly; X³ is Asp, or Glu; X⁵ is Ser, or Lys; X⁷ is Ser, or Lys; X⁸ is Asp, Glu, or Lys; X⁹ is Asp, Glu, or Lys; X¹⁰ is Met, Lys, Leu, Ile, or Nor-Leucine; X¹¹ is Asn, or Lys; X¹² is Thr, or Lys; X¹³ is Ile, or Lys; X¹⁴ is Leu, or Lys; X¹⁵ is Asp, or Lys; X¹⁶ is Asn, or Lys; X¹⁷ is Leu, or Lys; X¹⁸ is Ala, or Lys; X²⁰ is Arg, or Lys; X²¹ is Asp, or Lys; X²⁴ is Asn, or Lys; X²⁸ is Gln, or Lys; X³³ is Asp, Glu, Lys, Asp-Arg, or Asp-Lys.

In one embodiment of the invention the GLP-2 derivative comprises a GLP-2 peptide consisting of the amino acid sequence
His-X²-X³-Gly-X⁵-Phe-X⁷-X⁸-X⁹-X¹⁰-X¹¹-X¹²-X¹³-X¹⁴-X¹⁵-X¹⁶-X¹⁷-X¹⁸-Ala-X²⁰-X²¹-Phe-Ile-X²⁴-Trp-Leu-Ile-X²⁸-Thr-X³⁰-Ile-Thr-X³³
   or a fragment thereof; wherein X² is Ala, Val or Gly; X³ is Asp, or Glu; X⁵ is Ser, or Lys; X⁷ is Ser, or Lys; X⁸ is Asp, Glu, or Lys; X⁹ is Asp, Glu, or Lys; X¹⁰ is Met, Lys, Leu, Ile, or Nor-Leucine; X¹¹ is Asn, or Lys; X¹² is Thr, or Lys; X¹³ is Ile, or Lys; X¹⁴ is Leu, or Lys; X¹⁵ is Asp, or Lys; X¹⁶ is Asn, or Lys; X¹⁷ is Leu, or Lys; X¹⁸ is Ala, or Lys; X²⁰ is Arg, or Lys; X²¹ is Asp, or Lys; X²⁴ is Asn, or Lys; X²⁸ is Gln, or Lys; X³³ is Asp, Glu, Lys, Asp-Arg, or Asp-Lys. In one embodiment X² is Ala. In one embodiment X² is Gly. In one embodiment X³ is Asp. In one embodiment X³ is Glu. In one embodiment X⁵ is Ser. In one embodiment X⁷ is Ser. In one embodiment X⁸ is Asp. In one embodiment X⁸ is Glu. In one embodiment X⁹ is Asp. In one embodiment X⁹ is Glu. In one embodiment X¹⁰ is selected from the group consisting of Met, Leu, Ile, and Nor-Leucine. In one embodiment X¹¹ is Asn. In one embodiment X¹² is Thr. In one embodiment X¹³ is Ile. In one embodiment X¹⁴ is Leu. In one embodiment X¹⁵ is Asp. In one embodiment X¹⁶ is Asn. In one embodiment X¹⁷ is Leu. In one embodiment X¹⁸ is Ala. In one embodiment X²¹ is Asp. In one embodiment X²⁴ is Asn. In one embodiment X²⁸ is Gln. In one embodiment X³³ is Asp. In one embodiment X³³ is Glu. In one embodiment X³³ is Asp-Arg. In one embodiment one or more amino acid independently selected from the list consisting of is a Lys. In X⁵, X⁷, X⁸, X⁹, X¹⁰, X¹¹, X¹², X¹³, X¹⁴, X¹⁵, X¹⁶, X¹⁷, X¹⁸, X²⁰, X²¹, X²⁴, X²⁸, and X³³ is a Lys. In one embodiment one or more amino acid independently selected from the list consisting of X⁵, X⁷, X⁸, X⁹, X¹⁰, X¹¹, X¹², X¹³, X¹⁴, X¹⁵, X¹⁶, X¹⁷, X¹⁸, X²⁰, X²¹, X²⁴, X²⁸, and X³³ is a Lys. In one embodiment the amino acid X⁵ is Lys. In one embodiment the amino acid X⁷ is Lys. In one embodiment the amino acid X⁸ is Lys. In one embodiment the amino acid X⁹ is Lys. In one embodiment the amino acid X¹⁰ is Lys. In one embodiment the amino acid X¹¹ is Lys. In one embodiment the amino acid X¹² is Lys. In one embodiment the amino acid X¹³ is Lys. In one embodiment the amino acid X¹⁴ is Lys. In one embodiment the amino acid X¹⁵ is Lys. In one embodiment the amino acid X¹⁶ is Lys. In one embodiment the amino acid X¹⁷ is Lys. In one embodiment the amino acid X¹⁸ is Lys. In one embodiment the amino acid X²⁰ is Lys. In one embodiment the amino acid X²¹ is Lys. In one embodiment the amino acid X²⁴ is Lys. In one embodiment the amino acid X²⁸ is Lys. In one embodiment the amino acid X³³ is Lys. In one embodiment the amino acid X³³ is Asp-Arg.

In one embodiment of the invention the GLP-2 peptide is a GLP-2 peptide, wherein a total of up to 5 amino acid residues have been exchanged with any α-amino acid residue, such as 4 amino acid residues, 3 amino acid residues, 2 amino acid residues, or 1 amino acid residue.

In one embodiment of the invention the GLP-2 peptide is selected from the list consisting of: GLP-2(1-33), 34R-GLP-2(1-34), A2G-GLP-2(1-33), A2G/34R-GLP-2(1-34); K30R-GLP-2(1-33); S5K-GLP-2(1-33); S7K-GLP-2(1-33); D8K-GLP-2(1-33); E9K-GLP-2(1-33); M10K-GLP-2(1-33); N11K-GLP-2(1-33); T12K-GLP-2(1-33); I13K-GLP-2(1-33); L14K-GLP-2((1-33); D15K-GLP-2(1-33); N 16K-GLP-2(1-33); L17K-GLP-2(1-33); A18K-GLP-2(1-33); D21-K-GLP2(1-33); N24K-GLP-2(1-33); Q28K-GLP-2(1-33); S5K/K30R-GLP-2(1-33); S7K/K30R-GLP-2(1-33); D8K/K30R-GLP-2(1-33); E9K/K30R-GLP-2(1-33); M10K/K30R-GLP-2(1-33); N11K/K30R-GLP-2(1-33); T12K/K30R-GLP-2(1-33); I13K/K30R-GLP-2(1-33); L14K/K30R-GLP-2(1-33); D15K/K30R-GLP-2(1-33); N16K/K30R-GLP-2(1-33); L17K/K30R-GLP-2(1-33); A18K/K30R-GLP2(1-33); D21K/K30R-GLP-2(1-33); N24K/K30R-GLP-2(1-33); Q28K/K30R-GLP-2(1-33); K30R/D33K-GLP-2(1-33); D3E/K30R/D33E-GLP-2(1-33); D3E/S5K/K30R/D33E-GLP-2(1-33); D3E/S7K/K30R/D33E-GLP-2(1-33); D3E/D8K/K30R/D33E-GLP-2(1-33); D3E/E9K/K30R/D33E-GLP-2(1-33); D3E/M10K/K30R/D33E-GLP-2(1-33); D3E/N11 K/K30R/D33E-GLP-2(1-33); D3E/T12K/K30R/D33E-GLP-2(1-33); D3E/I13K/K30R/D33E-GLP-2(1-33); D3E/L14K/K30R/D33E-GLP-2(1-33); D3E/D15K/K30R/D33E-GLP-2(1-33); D3E/N16K/K30R/D33E-GLP-2(1-33); D3E/L17K/K30R/D33E-GLP-2(1-33); D3E/A18K/K30R/D33E-GLP-2(1-33); D3E/D21K/K30R/D33E-GLP-2(1-33); D3E/N24K/K30R/D33E-GLP-2(1-33); and D3E/Q28K/K30R/D33E-GLP-2(1-33).

In one embodiment of the invention the GLP-2 derivative only has one hydrophilic substituent attached to the GLP-2 peptide.

In one embodiment of the invention the hydrophilic substituent comprises H(OCH₂CH₂)ₙO— wherein n>4 with a molecular weight from about 200 to about 100.000 daltons.

In one embodiment of the invention the hydrophilic substituent comprises CH₃O—(CH₂CH₂O)ₙ—CH₂CH₂—O— wherein n>4 with a molecular weight from about 200 to about 100.000 Daltons.

In one embodiment of the invention the hydrophilic substituent is polyethylen glycol (PEG) with a molecular weight from about 200 to about 5000 Daltons.

In one embodiment of the invention the hydrophilic substituent is polyethylen glycol (PEG) with a molecular weight from about 5000 to about 20.000 Daltons.

In one embodiment of the invention the hydrophilic substituent is polyethylen glycol (PEG) with a molecular weight from about 20.000 to about 100.000 Daltons.

In one embodiment of the invention the hydrophilic substituent comprises is a methoxy-PEG (mPEG) with a molecular weight from about 200 to about 5000 Daltons.

In one embodiment of the invention the hydrophilic substituent is methoxy-polyethylen glycol (mPEG) with a molecular weight from about 5000 to about 20.000 Daltons.

In one embodiment of the invention the hydrophilic substituent is methoxy-polyethylen glycol (mPEG) with a molecular weight from about 20.000 to about 100.000 daltons.

In one embodiment of the invention the hydrophilic substituent is attached to an amino acid residue in such a way that a carboxyl group of the hydrophilic substituent forms an amide bond with an amino group of the amino acid residue.

In one embodiment of the invention the hydrophilic substituent is attached to a Lys residue.

In one embodiment of the invention the hydrophilic substituent is attached to an amino acid residue in such a way that an amino group of the hydrophilic substituent forms an amide bond with a carboxyl group of the amino acid residue.

In one embodiment of the invention the hydrophilic substituent is attached to the GLP-2 peptide by means of a spacer.

In one embodiment of the invention the spacer is an unbranched alkane α,ω-dicarboxylic acid group having from 1 to 7 methylene groups, such as two methylene groups which spacer forms a bridge between an amino group of the GLP-2 peptide and an amino group of the hydrophilic substituent In one embodiment of the invention the spacer is an amino acid residue except a Cys residue, or a dipeptide. Examples of suitable spacers includes P-alanine, gamma-aminobutyric acid (GABA), γ-glutamic acid, succinic acid, Lys, Glu or Asp, or a dipeptide such as Gly-Lys. When the spacer is succinic acid, one carboxyl group thereof may form an amide bond with an amino group of the amino acid residue, and the other carboxyl group thereof may form an amide bond with an amino group of the hydrophilic substituent. When the spacer is Lys, Glu or Asp, the carboxyl group thereof may form an amide bond with an amino group of the amino acid residue, and the amino group thereof may form an amide bond with a carboxyl group of the hydrophilic substituent. When Lys is used as the spacer, a further spacer may in some instances be inserted between the ε-amino group of Lys and the hydrophilic substituent. In one embodiment, such a further spacer is succinic acid which forms an amide bond with the ε-amino group of Lys and with an amino group present in the hydrophilic substituent . In another embodiment such a further spacer is Glu or Asp which forms an amide bond with the ε-amino group of Lys and another amide bond with a carboxyl group present in the hydrophilic substituent, that is, the hydrophilic substituent is a N-acylated lysine residue.

In one embodiment of the invention the spacer is selected from the list consisting of β-alanine, gamma-aminobutyric acid (GABA), γ-glutamic acid, Lys, Asp, Glu, a dipeptide containing Asp, a dipeptide containing Glu, or a dipeptide containing Lys. In one embodiment of the invention the spacer is β-alanine. In one embodiment of the invention the spacer is gamma-aminobutyric acid (GABA). In one embodiment of the invention the spacer is γ-glutamic acid.

In one embodiment of the invention a carboxyl group of the parent GLP-2 peptide forms an amide bond with an amino group of a spacer, and the carboxyl group of the amino acid or dipeptide spacer forms an amide bond with an amino group of the hydrophilic substituent.

In one embodiment of the invention an amino group of the parent GLP-2 peptide forms an amide bond with a carboxylic group of a spacer, and an amino group of the spacer forms an amide bond with a carboxyl group of the hydrophilic substituent.

In one embodiment of the invention the GLP-2 derivative has one hydrophilic substituent In one embodiment of the invention the GLP-2 derivative has two hydrophilic substituent. In one embodiment of the invention the GLP-2 derivative has three hydrophilic substituent. In one embodiment of the invention the GLP-2 derivative has four hydrophilic substituent.

In one embodiment of the invention the GLP-2 derivative is selected from the group consisting of
S5K(3-(PAO-amino)propionyl)-GLP-2(1-33);
S7K(3-(PAO-amino)propionyl)-GLP-2(1-33);
D8K(3-(PAO-amino)propionyl)-GLP-2(1-33);
E9K(3-(PAO-amino)propionyl)-GLP-2(1-33);
M10K(3-(PAO-amino)propionyl)-GLP-2(1-33);
N11K(3-(PAO-amino)propionyl)-GLP-2(1-33);
T12K(3-(PAO-amino)propionyl)-GLP-2(1-33);
I13K(3-(PAO-amino)propionyl)-GLP-2(1-33);
L14K(3-(PAO-amino)propionyl)-GLP-2(1-33);
D15K(3-(PAO-amino)propionyl)-GLP-2(1-33);
N16K(3-(PAO-amino)propionyl)-GLP-2(1-33);
L17K(3-(PAO-amino)propionyl)-GLP-2(1-33);
L17K((S)-4-carboxy-4-(PAO-amino)butanoyl)-GLP-2(1-33);
L17K(4-(PAO-amino)butanoyl)-GLP-2(1-33);
A18K(3-(PAO-amino)propionyl)-GLP-2(1-33);
D21K(3-(PAO-amino)propionyl)-GLP-2(1-33);
N24K(3-(PAO-amino)propionyl)-GLP-2(1-33);
Q28K(3-(PAO-amino)propionyl)-GLP-2(1-33);
S5K(3-(PAO-amino)propionyl)/K30R-GLP-2(1-33);
S7K(3-(PAO-amino)propionyl)/K30R-GLP-2(1-33);
D8K(3-(PAO-amino)propionyl)/K30R-GLP-2(1-33);
E9K(3-(PAO-amino)propionyl)/K30R-GLP-2(1-33);
M10K(3-(PAO-amino)propionyl)/K30R-GLP-2(1-33);
N11K(3-(PAO-amino)propionyl)/K30R-GLP-2(1-33);
T12K(3-(PAO-amino)propionyl)/K30R-GLP-2(1-33);
I13K(3-(PAO-amino)propionyl)/K30R-GLP-2(1-33);
L14K(3-(PAO-amino)propionyl)/K30R-GLP-2(1-33);
D15K(3-(PAO-amino)propionyl)/K30R-GLP-2(1-33);
N16K(3-(PAO-amino)propionyl)/K30R-GLP-2(1-33);
L17K(3-(PAO-amino)propionyl)/K30R-GLP-2(1-33);
L17K((S)-4-carboxy-4-(PAO-amino)butanoyl)/K30R-GLP-2(1-33);
L17K(4-(PAO-amino)butanoyl)/K30R-GLP-2(1-33);
A18K(3-(PAO-amino)propionyl)/K30R-GLP-2(1-33);
D21K(3-(PAO-amino)propionyl)/K30R-GLP-2(1-33);
N24K(3-(PAO-amino)propionyl)/K30R-GLP-2(1-33);
Q28K(3-(PAO-amino)propionyl)/K30R-GLP-2(1-33);
D3E/S5K(3-(PAO-amino)propionyl)/K30R/D33E-GLP-2(1-33);
D3E/S7K(3-(PAO-amino)propionyl)/K30R/D33E-GLP-2(1-33);
D3E/D8K(3-(PAO-amino)propionyl)/K30R/D33E-GLP-2(1-33);
D3E/E9K(3-(PAO-amino)propionyl)/K30R/D33E-GLP-2(1-33);
D3E/M10K(3-(PAO-amino)propionyl)/K30R/D33E-GLP-2(1-33);
D3E/N11K(3-(PAO-amino)propionyl)/K30R/D33E-GLP-2(1-33);
D3E/T12K(3-(PAO-amino)propionyl)/K30R/D33E-GLP-2(1-33);
D3E/I13K(3-(PAO-amino)propionyl)/K30R/D33E-GLP-2(1-33);
D3E/L14K(3-(PAO-amino)propionyl)/K30R/D33E-GLP-2(1-33);
D3E/D15K(3-(PAO-amino)propionyl)/K30R/D33E-GLP-2(1-33);
D3E/N16K(3-(PAO-amino)propionyl)/K30R/D33E-GLP-2(1-33);
D3E/L17K(3-(PAO-amino)propionyl)/K30R/D33E-GLP-2(1-33);
D3E/L17K((S)-4-carboxy-4-(PAO-amino)butanoyl)/K30R/D33E-GLP-2(1-33);
D3E/L17K(4-(PAO-amino)butanoyl)/K30R/D33E-GLP-2(1-33);
D3E/A18K(3-(PAO-amino)propionyl)/K30R/D33E-GLP-2(1-33);
D3E/D21K(3-(PAO-amino)propionyl)/K30R/D33E-GLP-2(1-33);
D3E/N24K(3-(PAO-amino)propionyl)/K30R/D33E-GLP-2(1-33);
D3E/Q28K(3-(PAO-amino)propionyl)/K30R/D33E-GLP-2(1-33);
S5K(N^{ε}-PAO)-GLP-2(1-33);
S7K(N^{ε}-PAO)-GLP-2(1-33);
D8K(N^{ε}-PAO)-GLP-2(1-33);
E9K(N^{ε}-PAO)-GLP-2(1-33);
M10K(N^{ε}-PAO)-GLP-2(1-33);
N11K(N^{ε}-PAO)-GLP-2(1-33);
T12K(N^{ε}-PAO)-GLP-2(1-33);
I13K(N^{ε}-PAO)-GLP-2(1-33);
L14K(N^{ε}-PAO)-GLP-2(1-33);
D15K(N^{ε}-PAO)-GLP-2(1-33);
N16K(N^{ε}-PAO)-GLP-2(1-33);
L17K(N^{ε}-PAO)-GLP-2(1-33);
A18K(N^{ε}-PAO)-GLP-2(1-33);
D21K(N^{ε}-PAO)-GLP-2(1-33);
N24K(N^{ε}-PAO)-GLP-2(1-33);
Q28K(N^{ε}-PAO)-GLP-2(1-33);
S5K(N^{ε}-PAO)/K30R-GLP-2(1-33);
S7K(N^{ε}-PAO)/K30R-GLP-2(1-33);
D8K(N^{ε}-PAO)/K30R-GLP-2(1-33);
E9K(N^{ε}-PAO)/K30R-GLP-2(1-33);
M10K(N^{ε}-PAO)/K30R-GLP-2(1-33);
N11K(N^{ε}-PAO)/K30R-GLP-2(1-33);
T12K(N^{ε}-PAO)/K30R-GLP-2(1-33);
I13K(N^{ε}-PAO)/K30R-GLP-2(1-33);
L14K(N^{ε}-PAO)/K30R-GLP-2(1-33);
D15K(N^{ε}-PAO)/K30R-GLP-2(1-33);
N16K(N^{ε}-PAO)/K30R-GLP-2(1-33);
L17K(N^{ε}-PAO)/K30R-GLP-2(1-33);
A18K(N^{ε}-PAO)propionyl)/K30R-GLP-2(1-33);
D21K(N^{ε}-PAO)/K30R-GLP-2(1-33);
N24K(N^{ε}-PAO)/K30R-GLP-2(1-33);
Q28K(N^{ε}-PAO)/K30R-GLP-2(1-33);
D3E/S5K(N^{ε}-PAO)/K30R/D33E-GLP-2(1-33);
D3E/S7K(3-(N^{ε}-PAO)/K30R/D33E-GLP-2(1-33);
D3E/D8K(N^{ε}-PAO)/K30R/D33E-GLP-2(1-33);
D3E/E9K(N^{ε}-PAO)/K30R/D33E-GLP-2(1-33);
D3E/M10K(N^{ε}-PAO)/K30R/D33E-GLP-2(1-33);
D3E/N11K(N^{ε}-PAO)/K30R/D33E-GLP-2(1-33);
D3E/T12K(N^{ε}-PAO)/K30R/D33E-GLP-2(1-33);
D3E/I13K(N^{ε}-PAO)/K30R/D33E-GLP-2(1-33);
D3E/L14K(N^{ε}-PAO)/K30R/D33E-GLP-2(1-33);
D3E/D15K(N^{ε}-PAO)/K30R/D33E-GLP-2(1-33);
D3E/N16K(N^{ε}-PAO)/K30R/D33E-GLP-2(1-33);
D3E/L17K(N^{ε}-PAO)/K30R/D33E-GLP-2(1-33);
D3E/A18K(N^{ε}-PAO)/K30R/D33E-GLP-2(1-33);
D3E/D21K(N^{ε}-PAO)/K30R/D33E-GLP-2(1-33);
D3E/N24K(N^{ε}-PAO)/K30R/D33E-GLP-2(1-33); and
D3E/Q28K(N^{ε}-PAO)/K30R/D33E-GLP-2(1-33).

In one embodiment of the invention the GLP-2 derivative is selected from the group consisting of
S5K(3-(PEG-amino)propionyl)-GLP-2(1-33);
S7K(3-(PEG-amino)propionyl)-GLP-2(1-33);
D8K(3-(PEG-amino)propionyl)-GLP-2(1-33);
E9K(3-(PEG-amino)propionyl)-GLP-2(1-33);
M10K(3-(PEG-amino)propionyl)-GLP-2(1-33);
N11K(3-(PEG-amino)propionyl)-GLP-2(1-33);
T12K(3-(PEG-amino)propionyl)-GLP-2(1-33);
I13K(3-(PEG-amino)propionyl)-GLP-2(1-33);
L14K(3-(PEG-amino)propionyl)-GLP-2(1-33);
D15K(3-(PEG-amino)propionyl)-GLP-2(1-33);
N16K(3-(PEG-amino)propionyl)-GLP-2(1-33);
L17K(3-(PEG-amino)propionyl)-GLP-2(1-33);
L17K((S)-4-carboxy-4-(PEG-amino)butanoyl)-GLP-2(1-33);
L17K(4-(PEG-amino)butanoyl)-GLP-2(1-33);
A18K(3-(PEG-amino)propionyl)-GLP-2(1-33);
D21K(3-(PEG-amino)propionyl)-GLP-2(1-33);
N24K(3-(PEG-amino)propionyl)-GLP-2(1-33);
Q28K(3-(PEG-amino)propionyl)-GLP-2(1-33);
S5K(3-(PEG-amino)propionyl)/K30R-GLP-2(1-33);
S7K(3-(PEG-amino)propionyl)/K30R-GLP-2(1-33);
D8K(3-(PEG-amino)propionyl)/K30R-GLP-2(1-33);
E9K(3-(PEG-amino)propionyl)/K30R-GLP-2(1-33);
M10K(3-(PEG-amino)propionyl)/K30R-GLP-2(1-33);
N11K(3-(PEG-amino)propionyl)/K30R-GLP-2(1-33);
T12K(3-(PEG-amino)propionyl)/K30R-GLP-2(1-33);
I13K(3-(PEG-amino)propionyl)/K30R-GLP-2(1-33);
L14K(3-(PEG-amino)propionyl)/K30R-GLP-2(1-33);
D15K(3-(PEG-amino)propionyl)/K30R-GLP-2(1-33);
N16K(3-(PEG-amino)propionyl)/K30R-GLP-2(1-33);
L17K(3-(PEG-amino)propionyl)/K30R-GLP-2(1-33);
L17K((S)-4-carboxy-4-(PEG-amino)butanoyl)/K30R-GLP-2(1-33);
L17K(4-(PEG-amino)butanoyl)/K30R-GLP-2(1-33);
A18K(3-(PEG-amino)propionyl)/K30R-GLP-2(1-33);
D21K(3-(PEG-amino)propionyl)/K30R-GLP-2(1-33);
N24K(3-(PEG-amino)propionyl)/K30R-GLP-2(1-33);
Q28K(3-(PEG-amino)propionyl)/K30R-GLP-2(1-33);
D3E/S5K(3-(PEG-amino)propionyl)/K30R/D33E-GLP-2(1-33);
D3E/S7K(3-(PEG-amino)propionyl)/K30R/D33E-GLP-2(1-33);
D3E/D8K(3-(PEG-amino)propionyl)/K30R/D33E-GLP-2(1-33);
D3E/E9K(3-(PEG-amino)propionyl)/K30R/D33E-GLP-2(1-33);
D3E/M10K(3-(PEG-amino)propionyl)/K30R/D33E-GLP-2(1-33);
D3E/N11K(3-(PEG-amino)propionyl)/K30R/D33E-GLP-2(1-33);
D3E/T12K(3-(PEG-amino)propionyl)/K30R/D33E-GLP-2(1-33);
D3E/I13K(3-(PEG-amino)propionyl)/K30R/D33E-GLP-2(1-33);
D3E/L14K(3-(PEG-amino)propionyl)/K30R/D33E-GLP-2(1-33);
D3E/D15K(3-(PEG-amino)propionyl)/K30R/D33E-GLP-2(1-33);
D3E/N16K(3-(PEG-amino)propionyl)/K30R/D33E-GLP-2(1-33);
D3E/L17K(3-(PEG-amino)propionyl)/K30R/D33E-GLP-2(1-33);
D3E/L17K((S)-4-carboxy-4-(PEG-amino)butanoyl)/K30R/D33E-GLP-2(1-33);
D3E/L17K(4-(PEG-amino)butanoyl)/K30R/D33E-GLP-2(1-33);
D3E/A18K(3-(PEG-amino)propionyl)/K30R/D33E-GLP-2(1-33);
D3E/D21K(3-(PEG-amino)propionyl)/K30R/D33E-GLP-2(1-33);
D3E/N24K(3-(PEG-amino)propionyl)/K30R/D33E-GLP-2(1-33);
D3E/Q28K(3-(PEG-amino)propionyl)/K30R/D33E-GLP-2(1-33);
S5K(N^{ε}-PEG)-GLP-2(1-33);
S7K(N^{ε}-PEG)-GLP-2(1-33);
D8K(N^{ε}-PEG)-GLP-2(1-33);
E9K(N^{ε}-PEG)-GLP-2(1-33);
M10K(N^{ε}-PEG)-GLP-2(1-33);
N11K(N^{ε}-PEG)-GLP-2(1-33);
T12K(N^{ε}-PEG)-GLP-2(1-33);
I13K(N^{ε}-PEG)-GLP-2(1-33);
L14K(N^{ε}-PEG)-GLP-2(1-33);
D15K(N^{ε}-PEG)-GLP-2(1-33);
N16K(N^{ε}-PEG)-GLP-2(1-33);
L17K(N^{ε}-PEG)-GLP-2(1-33);
A18K(N^{ε}-PEG)-GLP-2(1-33);
D21K(N^{ε}-PEG)-GLP-2(1-33);
N24K(N^{ε}-PEG)-GLP-2(1-33);
Q28K(N^{ε}-PEG)-GLP-2(1-33);
S5K(N^{ε}-PEG)/K30R-GLP-2(1-33);
S7K(N^{ε}-PEG)/K30R-GLP-2(1-33);
D8K(N^{ε}-PEG)/K30R-GLP-2(1-33);
E9K(N^{ε}-PEG)/K30R-GLP-2(1-33);
M10K(N^{ε}-PEG)/K30R-GLP-2(1-33);
N11K(N^{ε}-PEG)/K30R-GLP-2(1-33);
T12K(N^{ε}-PEG)/K30R-GLP-2(1-33);
I13K(N^{ε}-PEG)/K30R-GLP-2(1-33);
L14K(N^{ε}-PEG)/K30R-GLP-2(1-33);
D15K(N^{ε}-PEG)/K30R-GLP-2(1-33);
N16K(N^{ε}-PEG)/K30R-GLP-2(1-33);
L17K(N^{ε}-PEG)/K30R-GLP-2(1-33);
A18K(N^{ε}-PEG)propionyl)/K30R-GLP-2(1-33);
D21K(N^{ε}-PEG)/K30R-GLP-2(1-33);
N24K(N^{ε}-PEG)/K30R-GLP-2(1-33);
Q28K(N^{ε}-PEG)/K30R-GLP-2(1-33);
D3E/S5K(N^{ε}-PEG)/K30R/D33E-GLP-2(1-33);
D3E/S7K(3-(N^{ε}-PEG)/K30R/D33E-GLP-2(1-33);
D3E/D8K(N^{ε}-PEG)/K30R/D33E-GLP-2(1-33);
D3E/E9K(N^{ε}-PEG)/K30R/D33E-GLP-2(1-33);
D3E/M10K(N^{ε}-PEG)/K30R/D33E-GLP-2(1-33);
D3E/N11K(N^{ε}-PEG)/K30R/D33E-GLP-2(1-33);
D3E/T12K(N^{ε}-PEG)/K30R/D33E-GLP-2(1-33);
D3E/I13K(N^{ε}-PEG)/K30R/D33E-GLP-2(1-33);
D3E/L14K(N^{ε}-PEG)/K30R/D33E-GLP-2(1-33);
D3E/D15K(N^{ε}-PEG)/K30R/D33E-GLP-2(1-33);
D3E/N16K(N^{ε}-PEG)/K30R/D33E-GLP-2(1-33);
D3E/L17K(N^{ε}-PEG)/K30R/D33E-GLP-2(1-33);
D3E/A18K(N^{ε}-PEG)/K30R/D33E-GLP-2(1-33);
D3E/D21K(N^{ε}-PEG)/K30R/D33E-GLP-2(1-33);
D3E/N24K(N^{ε}-PEG)/K30R/D33E-GLP-2(1-33); and
D3E/Q28K(N^{ε}-PEG)/K30R/D33E-GLP-2(1-33), wherein the PEG substituent in any of the compounds has a molecular weight from about 200 to about 100.000 Daltons.

In one embodiment of the invention the GLP-2 derivative is selected from the group consisting of
A2G/S5K(3-(PAO-amino)propionyl)-GLP-2(1-33);
A2G/S7K(3-(PAO-amino)propionyl)-GLP-2(1-33);
A2G/D8K(3-(PAO-amino)propionyl)-GLP-2(1-33);
A2G/E9K(3-(PAO-amino)propionyl)-GLP-2(1-33);
A2G/M10K(3-(PAO-amino)propionyl)-GLP-2(1-33);
A2G/N11K(3-(PAO-amino)propionyl)-GLP-2(1-33);
A2G/T12K(3-(PAO-amino)propionyl)-GLP-2(1-33);
A2G/I13K(3-(PAO-amino)propionyl)-GLP-2(1-33);
A2G/L14K(3-(PAO-amino)propionyl)-GLP-2(1-33);
A2G/D15K(3-(PAO-amino)propionyl)-GLP-2(1-33);
A2G/N16K(3-(PAO-amino)propionyl)-GLP-2(1-33);
A2G/L17K(3-(PAO-amino)propionyl)-GLP-2(1-33);
A2G/L17K((S)-4-carboxy-4-(PAO-amino)butanoyl)-GLP-2(1-33);
A2G/L17K(4-(PAO-amino)butanoyl)-GLP-2(1-33);
A2G/A18K(3-(PAO-amino)propionyl)-GLP-2(1-33);
A2G/D21K(3-(PAO-amino)propionyl)-GLP-2(1-33);
A2G/N24K(3-(PAO-amino)propionyl)-GLP-2(1-33);
A2G/Q28K(3-(PAO-amino)propionyl)-GLP-2(1-33);
A2G/S5K(3-(PAO-amino)propionyl)/K30R-GLP-2(1-33);
A2G/S7K(3-(PAO-amino)propionyl)/K30R-GLP-2(1-33);
A2G/D8K(3-(PAO-amino)propionyl)/K30R-GLP-2(1-33);
A2G/E9K(3-(PAO-amino)propionyl)/K30R-GLP-2(1-33);
A2G/M10K(3-(PAO-amino)propionyl)/K30R-GLP-2(1-33);
A2G/N11K(3-(PAO-amino)propionyl)/K30R-GLP-2(1-33);
A2G/T12K(3-(PAO-amino)propionyl)/K30R-GLP-2(1-33);
A2G/I13K(3-(PAO-amino)propionyl)/K30R-GLP-2(1-33);
A2G/L14K(3-(PAO-amino)propionyl)/K30R-GLP-2(1-33);
A2G/D15K(3-(PAO-amino)propionyl)/K30R-GLP-2(1-33);
A2G/N16K(3-(PAO-amino)propionyl)/K30R-GLP-2(1-33);
A2G/L17K(3-(PAO-amino)propionyl)/K30R-GLP-2(1-33);
A2G/L17K((S)-4-carboxy-4-(PAO-amino)butanoyl)/K30R-GLP-2(1-33);
A2G/L17K(4-(PAO-amino)butanoyl)/K30R-GLP-2(1-33);
A2G/A18K(3-(PAO-amino)propionyl)/K30R-GLP-2(1-33);
A2G/D21K(3-(PAO-amino)propionyl)/K30R-GLP-2(1-33);
A2G/N24K(3-(PAO-amino)propionyl)/K30R-GLP-2(1-33);
A2G/Q28K(3-(PAO-amino)propionyl)/K30R-GLP-2(1-33);
A2G/D3E/S5K(3-(PAO-amino)propionyl)/K30R/D33E-GLP-2(1-33);
A2G/D3E/S7K(3-(PAO-amino)propionyl)/K30R/D33E-GLP-2(1-33);
A2G/D3E/D8K(3-(PAO-amino)propionyl)/K30R/D33E-GLP-2(1-33);
A2G/D3E/E9K(3-(PAO-amino)propionyl)/K30R/D33E-GLP-2(1-33);
A2G/D3E/M10K(3-(PAO-amino)propionyl)/K30R/D33E-GLP-2(1-33);
A2G/D3E/N11K(3-(PAO-amino)propionyl)/K30R/D33E-GLP-2(1-33);
A2G/D3E/T12K(3-(PAO-amino)propionyl)/K30R/D33E-GLP-2(1-33);
A2G/D3E/I13K(3-(PAO-amino)propionyl)/K30R/D33E-GLP-2(1-33);
A2G/D3E/L14K(3-(PAO-amino)propionyl)/K30R/D33E-GLP-2(1-33);
A2G/D3E/D15K(3-(PAO-amino)propionyl)/K30R/D33E-GLP-2(1-33);
A2G/D3E/N16K(3-(PAO-amino)propionyl)/K30R/D33E-GLP-2(1-33);
A2G/D3E/L17K(3-(PAO-amino)propionyl)/K30R/D33E-GLP-2(1-33);
A2G/D3E/L17K((S)-4-carboxy-4-(PAO-amino)butanoyl)/K30RID33E-GLP-2(1-33);
A2G/D3E/L17K(4-(PAO-amino)butanoyl)/K30R/D33E-GLP-2(1-33);
A2G/D3E/A18K(3-(PAO-amino)propionyl)/K30R/D33E-GLP-2(1-33);
A2G/D3E/D21K(3-(PAO-amino)propionyl)/K30R/D33E-GLP-2(1-33);
A2G/D3E/N24K(3-(PAO-amino)propionyl)/K30R/D33E-GLP-2(1-33);
A2G/D3E/Q28K(3-(PAO-amino)propionyl)/K30R/D33E-GLP-2(1-33);
A2G/S5K(N^{ε}-PAO)-GLP-2(1-33);
A2G/S7K(N^{ε}-PAO)-GLP-2(1-33);
A2G/D8K(N^{ε}-PAO)-GLP-2(1-33);
A2G/E9K(N^{ε}-PAO)-GLP-2(1-33);
A2G/M10K(N^{ε}-PAO)-GLP-2(1-33);
A2G/N11K(N^{ε}-PAO)-GLP-2(1-33);
A2G/T12K(N^{ε}-PAO)-GLP-2(1-33);
A2G/I13K(N^{ε}-PAO)-GLP-2(1-33);
A2G/L14K(N^{ε}-PAO)-GLP-2(1-33);
A2G/D15K(N^{ε}-PAO)-GLP-2(1-33);
A2G/N16K(N^{ε}-PAO)-GLP-2(1-33);
A2G/L17K(N^{ε}-PAO)-GLP-2(1-33);
A2G/A18K(N^{ε}-PAO)-GLP-2(1-33);
A2G/D21K(N^{ε}-PAO)-GLP-2(1-33);
A2G/N24K(N^{ε}-PAO)-GLP-2(1-33);
A2G/Q28K(N^{ε}-PAO)GLP-2(1-33);
A2G/S5K(N^{ε}-PAO)/K30R-GLP-2(1-33);
A2G/S7K(N^{ε}-PAO)/K30R-GLP-2(1-33);
A2G/D8K(N^{ε}-PAO)/K30R-GLP-2(1-33);
A2G/E9K(N^{ε}-PAO)/K30R-GLP-2(1-33);
A2G/M10K(N^{ε}-PAO)/K30R-GLP-2(1-33);
A2G/N11K(N^{ε}-PAO)/K30R-GLP-2(1-33);
A2G/T12K(N^{ε}-PAO)/K30R-GLP-2(1-33);
A2G/I13K(N^{ε}-PAO)/K30R-GLP-2(1-33);
A2G/L14K(N^{ε}-PAO)/K30R-GLP-2(1-33);
A2G/D15K(N^{ε}-PAO)/K30R-GLP-2(1-33);
A2G/N16K(N^{ε}-PAO)/K30R-GLP-2(1-33);
A2G/L17K(N^{ε}-PAO)/K30R-GLP-2(1-33);
A2G/A18K(N^{ε}-PAO)propionyl)/K30R-GLP-2(1-33);
A2G/D21K(N^{ε}-PAO)/K30R-GLP-2(1-33);
A2G/N24K(N^{ε}-PAO)/K30R-GLP-2(1-33);
A2G/Q28K(N^{ε}-PAO)/K30R-GLP-2(1-33);
A2G/D3E/S5K(N^{ε}-PAO)/K30R/D33E-GLP-2(1-33);
A2G/D3E/S7K(3-(N^{ε}-PAO)/K30R/D33E-GLP-2(1-33);
A2G/D3E/D8K(N^{ε}-PAO)/K30R/D33E-GLP-2(1-33);
A2G/D3E/E9K(N^{ε}-PAO)/K30R/D33E-GLP-2(1-33);
A2G/D3E/M10K(N^{ε}-PAO)/K30R/D33E-GLP-2(1-33);
A2G/D3E/N11K(N^{ε}-PAO)/K30R/D33E-GLP-2(1-33);
A2G/D3E/F12K(N^{ε}-PAO)/K30R/D33E-GLP-2(1-33);
A2G/D3E/I13K(N^{ε}-PAO)/K30R/D33E-GLP-2(1-33);
A2G/D3E/L14K(N^{ε}-PAO)/K30R/D33E-GLP-2(1-33);
A2G/D3E/D15K(N^{ε}-PAO)/K30R/D33E-GLP-2(1-33);
A2G/D3E/N16K(N^{ε}-PAO)/K30R/D33E-GLP-2(1-33);
A2G/D3E/L17K(N^{ε}-PAO)/K30R/D33E-GLP-2(1-33);
A2G/D3E/A18K(N^{ε}-PAO)/K30R/D33E-GLP-2(1-33);
A2G/D3E/D21K(N^{ε}-PAO)/K30R/D33E-GLP-2(1-33);
A2G/D3E/N24K(N^{ε}-PAO)/K30R/D33E-GLP-2(1-33); and
A2G/D3E/Q28K(N^{ε}-PAO)/K30R/D33E-GLP-2(1-33).

In one embodiment of the invention the GLP-2 derivative is selected from the group consisting of
A2G/S5K(3-(PEG-amino)propionyl)-GLP-2(1-33);
A2G/S7K(3-(PEG-amino)propionyl)-GLP-2(1-33);
A2G/D8K(3-(PEG-amino)propionyl)-GLP-2(1-33);
A2G/E9K(3-(PEG-amino)propionyl)-GLP-2(1-33);
A2G/M10K(3-(PEG-amino)propionyl)-GLP-2(1-33);
A2G/N11K(3-(PEG-amino)propionyl)-GLP-2(1-33);
A2G/T12K(3-(PEG-amino)propionyl)-GLP-2(1-33);
A2G/I13K(3-(PEG-amino)propionyl)-GLP-2(1-33);
A2G/L14K(3-(PEG-amino)propionyl)-GLP-2(1-33);
A2G/D15K(3-(PEG-amino)propionyl)-GLP-2(1-33);
A2G/N16K(3-(PEG-amino)propionyl)-GLP-2(1-33);
A2G/L17K(3-(PEG-amino)propionyl)-GLP-2(1-33);
A2G/L17K((S)-4-carboxy-4-(PEG-amino)butanoyl)-GLP-2(1-33);
A2G/L17K(4-(PEG-amino)butanoyl)-GLP-2(1-33);
A2G/A18K(3-(PEG-amino)propionyl)-GLP-2(1-33);
A2G/D21K(3-(PEG-amino)propionyl)-GLP-2(1-33);
A2G/N24K(3-(PEG-amino)propionyl)-GLP-2(1-33);
A2G/Q28K(3-(PEG-amino)propionyl)-GLP-2(1-33);
A2G/S5K(3-(PEG-amino)propionyl)/K30R-GLP-2(1-33);
A2G/S7K(3-(PEG-amino)propionyl)/K30R-GLP-2(1-33);
A2G/D8K(3-(PEG-amino)propionyl)/K30R-GLP-2(1-33);
A2G/E9K(3-(PEG-amino)propionyl)/K30R-GLP-2(1-33);
A2G/M10K(3-(PEG-amino)propionyl)/K30R-GLP-2(1-33);
A2G/N11K(3-(PEG-amino)propionyl)/K30R-GLP-2(1-33);
A2G/T12K(3-(PEG-amino)propionyl)/K30R-GLP-2(1-33);
A2G/I13K(3-(PEG-amino)propionyl)/K30R-GLP-2(1-33);
A2G/L14K(3-(PEG-amino)propionyl)/K30R-GLP-2(1-33);
A2G/D15K(3-(PEG-amino)propionyl)/K30R-GLP-2(1-33);
A2G/N16K(3-(PEG-amino)propionyl)/K30R-GLP-2(1-33);
A2G/L17K(3-(PEG-amino)propionyl)/K30R-GLP-2(1-33);
A2G/L17K((S)-4-carboxy-4-(PEG-amino)butanoyl)/K30R-GLP-2(1-33);
A2G/L17K(4-(PEG-amino)butanoyl)/K30R-GLP-2(1-33);
A2G/A18K(3-(PEG-amino)propionyl)/K30R-GLP-2(1-33);
A2G/D21K(3-(PEG-amino)propionyl)/K30R-GLP-2(1-33);
A2G/N24K(3-(PEG-amino)propionyl)/K30R-GLP-2(1-33);
A2G/Q28K(3-(PEG-amino)propionyl)/K30R-GLP-2(1-33);
A2G/D3E/S5K(3-(PEG-amino)propionyl)/K30R/D33E-GLP-2(1-33);
A2G/D3E/S7K(3-(PEG-amino)propionyl)/K30R/D33E-GLP-2(1-33);
A2G/D3E/D8K(3-(PEG-amino)propionyl)/K30R/D33E-GLP-2(1-33);
A2G/D3E/E9K(3-(PEG-amino)propionyl)/K30R/D33E-GLP-2(1-33);
A2G/D3E/M10K(3-(PEG-amino)propionyl)/K30R/D33E-GLP-2(1-33);
A2G/D3E/N11K(3-(PEG-amino)propionyl)/K30R/D33E-GLP-2(1-33);
A2G/D3E/T12K(3-(PEG-amino)propionyl)/K30R/D33E-GLP-2(1-33);
A2G/D3E/I13K(3-(PEG-amino)propionyl)/K30R/D33E-GLP-2(1-33);
A2G/D3E/L14K(3-(PEG-amino)propionyl)/K30R/D33E-GLP-2(1-33);
A2G/D3E/D15K(3-(PEG-amino)propionyl)/K30R/D33E-GLP-2(1-33);
A2G/D3E/N16K(3-(PEG-amino)propionyl)/K30R/D33E-GLP-2(1-33);
A2G/D3E/L17K(3-(PEG-amino)propionyl)/K30R/D33E-GLP-2(1-33);
A2G/D3E/L17K((S)-4-carboxy-4-(PEG-amino)butanoyl)/K30R/D33E-GLP-2(1-33);
A2G/D3E/L17K(4-(PEG-amino)butanoyl)/K30R/D33E-GLP-2(1-33);
A2G/D3E/A18K(3-(PEG-amino)propionyl)/K30R/D33E-GLP-2(1-33);
A2G/D3E/D21K(3-(PEG-amino)propionyl)/K30R/D33E-GLP-2(1-33);
A2G/D3E/N24K(3-(PEG-amino)propionyl)/K30R/D33E-GLP-2(1-33);
A2G/D3E/Q28K(3-(PEG-amino)propionyl)/K30R/D33E-GLP-2(1-33);
A2G/S5K(N^{ε}-PEG)-GLP-2(1-33);
A2G/S7K(N^{ε}-PEG)-GLP-2(1-33);
A2G/D8K(N^{ε}-PEG)-GLP-2(1-33);
A2G/E9K(N^{ε}-PEG)-GLP-2(1-33);
A2G/M10K(N^{ε}-PEG)-GLP-2(1-33);
A2G/N11K(N^{ε}-PEG)-GLP-2(1-33);
A2G/T12K(N^{ε}-PEG)-GLP-2(1-33);
A2G/I13K(N^{ε}-PEG)-GLP-2(1-33);
A2G/L14K(N^{ε}-PEG)-GLP-2(1-33);
A2G/D15K(N^{ε}-PEG)-GLP-2(1-33);
A2G/N16K(N^{ε}-PEG)-GLP-2(1-33);
A2G/L17K(N^{ε}-PEG)-GLP-2(1-33);
A2G/A18K(N^{ε}-PEG)-GLP-2(1-33);
A2G/D21K(N^{ε}-PEG)-GLP-2(1-33);
A2G/N24K(N^{ε}-PEG)-GLP-2(1-33);
A2G/Q28K(N^{ε}-PEG)-GLP-2(1-33);
A2G/S5K(N^{ε}-PEG)/K30R-GLP-2(1-33);
A2G/S7K(N^{ε}-PEG)/K30R-GLP-2(1-33);
A2G/D8K(N^{ε}-PEG)/K30R-GLP-2(1-33);
A2G/E9K(N^{ε}-PEG)/K30R-GLP-2(1-33);
A2G/M10K(N^{ε}-PEG)/K30R-GLP-2(1-33);
A2G/N11K(N^{ε}-PEG)/K30R-GLP-2(1-33);
A2G/T12K(N^{ε}-PEG)/K30R-GLP-2(1-33);
A2G/I13K(N^{ε}-PEG)/K30R-GLP-2(1-33);
A2G/L14K(N^{ε}-PEG)/K30R-GLP-2(1-33);
A2G/D15K(N^{ε}-PEG)/K30R-GLP-2(1-33);
A2G/N16K(N^{ε}-PEG)/K30R-GLP-2(1-33);
A2G/L17K(N^{ε}-PEG)/K30R-GLP-2(1-33);
A2G/A18K(N^{ε}-PEG)propionyl)/K30R-GLP-2(1-33);
A2G/D21K(N^{ε}-PEG)/K30R-GLP-2(1-33);
A2G/N24K(N^{ε}-PEG)/K30R-GLP-2(1-33);
A2G/Q28K(N^{ε}-PEG)/K30R-GLP-2(1-33);
A2G/D3E/S5K(N^{ε}-PEG)/K30R/D33E-GLP-2(1-33);
A2G/D3E/S7K(3-(N^{ε}-PEG)/K30R/D33E-GLP-2(1-33);
A2G/D3E/D8K(N^{ε}-PEG)/K30R/D33E-GLP-2(1-33);
A2G/D3E/E9K(N^{ε}-PEG)/K30R/D33E-GLP-2(1-33);
A2G/D3E/M10K(N^{ε}-PEG)/K30R/D33E-GLP-2(1-33);
A2G/D3E/N11K(N^{ε}-PEG)/K30R/D33E-GLP-2(1-33);
A2G/D3E/T12K(N^{ε}-PEG)/K30R/D33E-GLP-2(1-33);
A2G/D3E/I13K(N^{ε}-PEG)/K30R/D33E-GLP-2(1-33);
A2G/D3E/L14K(N^{ε}-PEG)/K30R/D33E-GLP-2(1-33);
A2G/D3E/D15K(N^{ε}-PEG)/K30R/D33E-GLP-2(1-33);
A2G/D3E/N16K(N^{ε}-PEG)/K30R/D33E-GLP-2(1-33);
A2G/D3E/L17K(N^{ε}-PEG)/K30R/D33E-GLP-2(1-33);
A2G/D3E/A18K(N^{ε}-PEG)/K30R/D33E-GLP-2(1-33);
A2G/D3E/D21K(N^{ε}-PEG)/K30R/D33E-GLP-2(1-33);
A2G/D3E/N24K(N^{ε}-PEG)/K30R/D33E-GLP-2(1-33); and
A2G/D3E/Q28K(N^{ε}-PEG)/K30R/D33E-GLP-2(1-33), wherein the PEG substituent in any of the compounds has a molecular weight from about 200 to about 100.000 Daltons.

In one embodiment of the invention the GLP-2 derivative is selected from the group consisting of
A2G/S5K(3-(PAO-amino)propionyl)/34R-GLP-2(1-34);
A2G/S7K(3-(PAO-amino)propionyl)/34R-GLP-2(1-34);
A2G/D8K(3-(PAO-amino)propionyl)/34R-GLP-2(1-34);
A2G/E9K(3-(PAO-amino)propionyl)/34R-GLP-2(1-34);
A2G/M10K(3-(PAO-amino)propionyl)/34R-GLP-2(1-34);
A2G/N11K(3-(PAO-amino)propionyl)/34R-GLP-2(1-34);
A2G/T12K(3-(PAO-amino)propionyl)/34R-GLP-2(1-34);
A2G/I13K(3-(PAO-amino)propionyl)/34R-GLP-2(1-34);
A2G/L14K(3-(PAO-amino)propionyl)/34R-GLP-2(1-34);
A2G/D15K(3-(PAO-amino)propionyl)/34R-GLP-2(1-34);
A2G/N16K(3-(PAO-amino)propionyl)/34R-GLP-2(1-34);
A2G/L17K(3-(PAO-amino)propionyl)/34R-GLP-2(1-34);
A2G/L17K((S)-4-carboxy-4-(PAO-amino)butanoyl )/34R-GLP-2(1-34);
A2G/L17K(4-(PAO-amino)butanoyl)/34R-GLP-2(1-34);
A2G/A18K(3-(PAO-amino)propionyl)/34R-GLP-2(1-34);
A2G/D21K(3-(PAO-amino)propionyl)/34R-GLP-2(1-34);
A2G/N24K(3-(PAO-amino)propionyl)/34R-GLP-2(1-34);
A2G/Q28K(3-(PAO-amino)propionyl)/34R-GLP-2(1-34);
A2G/S5K(3-(PAO-amino)propionyl)/K30R/34R-GLP-2(1-34);
A2G/S7K(3-(PAO-amino)propionyl)/K30R/34R-GLP-2(1-34);
A2G/D8K(3-(PAO-amino)propionyl)/K30R/34R-GLP-2(1-34);
A2G/E9K(3-(PAO-amino)propionyl)/K30R/34R-GLP-2(1-34);
A2G/M10K(3-(PAO-amino)propionyl)/K30R/34R-GLP-2(1.-34);
A2G/N11K(3-(PAO-amino)propionyl)/K30R/34R-GLP-2(1-34);
A2G/T12K(3-(PAO-amino)propionyl)/K30R/34R-GLP-2(1-34);
A2G/I13K(3-(PAO-amino)propionyl)/K30R/34R-GLP-2(1-34);
A2G/L14K(3-(PAO-amino)propionyl)/K30R/34R-GLP-2(1-34);
A2G/D15K(3-(PAO-amino)propionyl)/K30R/34R-GLP-2(1-34);
A2G/N16K(3-(PAO-amino)propionyl)/K30R/34R-GLP-2(1-34);
A2G/L17K(3-(PAO-amino)propionyl)/K30R/34R-GLP-2(1-34);
A2G/L17K((S)-4-carboxy-4-(PAO-amino)butanoyl)/K30R/34R-GLP-2(1-34);
A2G/L17K(4-(PAO-amino)butanoyl)/K30R/34R-GLP-2(1-34);
A2G/A18K(3-(PAO-amino)propionyl)/K30R/34R-GLP-2(1-34);
A2G/D21K(3-(PAO-amino)propionyl)/K30R/34R-GLP-2(1-34);
A2G/N24K(3-(PAO-amino)propionyl)/K30R/34R-GLP-2(1-34);
A2G/Q28K(3-(PAO-amino)propionyl)/K30R/34R-GLP-2(1-34);
A2G/D3E/S5K(3-(PAO-amino)propionyl)/K30R/D33E/34R-GLP-2(1-34);
A2G/D3E/S7K(3-(PAO-amino)propionyl)/K30R/D33E/34R-GLP-2(1-34);
A2G/D3E/D8K(3-(PAO-amino)propionyl)/K30R/D33E/34R-GLP-2(1-34);
A2G/D3E/E9K(3-(PAO-amino)propionyl)/K30R/D33E/34R-GLP-2(1-34);
A2G/D3E/M10K(3-(PAO-amino)propionyl)/K30R/D33E/34R-GLP-2(1-34);
A2G/D3E/N11K(3-(PAO-amino)propionyl)/K30R/D33E/34R-GLP-2(1-34);
A2G/D3E/T12K(3-(PAO-amino)propionyl)/K30R/D33E/34R-GLP-2(1-34);
A2G/D3E/I13K(3-(PAO-amino)propionyl)/K30R/D33E/34R-GLP-2(1-34);
A2G/D3E/L14K(3-(PAO-amino)propionyl)/K30R/D33E/34R-GLP-2(1-34);
A2G/D3E/D15K(3-(PAO-amino)propionyl)/K30R/D33E/34R-GLP-2(1-34);
A2G/D3E/N16K(3-(PAO-amino)propionyl)/K30R/D33E/34R-GLP-2(1-34);
A2G/D3E/L17K(3-(PAO-amino)propionyl)/K30R/D33E/34R-GLP-2(1-34);
A2G/D3E/L17K((S)-4-carboxy-4-(PAO-amino)butanoyl)/K30R/D33E/34R-GLP-2(1-34);
A2G/D3E/L17K(4-(PAO-amino)butanoyl)/K30R/D33E/34R-GLP-2(1-34);
A2G/D3E/A18K(3-(PAO-amino)propionyl)/K30R/D33E/34R-GLP-2(1-34);
A2G/D3E/D21K(3-(PAO-amino)propionyl)/K30R/D33E/34R-GLP-2(1-34);
A2G/D3E/N24K(3-(PAO-amino)propionyl)/K30R/D33E/34R-GLP-2(1-34);
A2G/D3E/Q28K(3-(PAO-amino)propionyl)/K3OR/D33E/34R-GLP-2(1-34);
A2G/S5K(N^{ε}-PAO)/34R-GLP-2(1-34);
A2G/S7K(N^{ε}-PAO)/34R-GLP-2(1-34);
A2G/D8K(N^{ε}-PAO)/34R-GLP-2(1-34);
A2G/E9K(N^{ε}-PAO)/34R-GLP-2(1-34);
A2G/M10K(N^{ε}-PAO)/34R-GLP-2(1-34);
A2G/N11K(N^{ε}-PAO)/34R-GLP-2(1-34);
A2G/T12K(N^{ε}-PAO)/34R-GLP-2(1-34);
A2G/I13K(N^{ε}-PAO)/34R-GLP-2(1-34);
A2G/L14K(N^{ε}-PAO)/34R-GLP-2(1-34);
A2G/D15K(N^{ε}-PAO)/34R-GLP-2(1-34);
A2G/N16K(N^{ε}-PAO)/34R-GLP-2(1-34);
A2G/L17K(N^{ε}-PAO)/34R-GLP-2(1-34);
A2G/A18K(N^{ε}-PAO)/34R-GLP-2(1-34);
A2G/D21K(N^{ε}-PAO)/34R-GLP-2(1-34);
A2G/N24K(N^{ε}-PAO)/34R-GLP-2(1-34);
A2G/Q28K(N^{ε}-PAO)/34R-GLP-2(1-34);
A2G/S5K(N^{ε}-PAO)/K30R/34R-GLP-2(1-34);
A2G/S7K(N^{ε}-PAO)/K30R/34R-GLP-2(1-34);
A2G/D8K(N^{ε}-PAO)/K30R/34R-GLP-2(1-34);
A2G/E9K(N^{ε}-PAO)/K30R/34R-GLP-2(1-34);
A2G/M10K(N^{ε}-PAO)/K30R/34R-GLP-2(1-34);
A2G/N11K(N^{ε}-PAO)/K30R/34R-GLP-2(1-34);
A2G/T12K(N^{ε}-PAO)/K30R/34R-GLP-2(1-34);
A2G/I13K(N^{ε}-PAO)/K30R/34R-GLP-2(1-34);
A2G/L14K(N^{ε}-PAO)/K30R/34R-GLP-2(1-34);
A2G/D15K(N^{ε}-PAO)/K30R/34R-GLP-2(1-34);
A2G/N16K(N^{ε}-PAO)/K30R/34R-GLP-2(1-34);
A2G/L17K(N^{ε}-PAO)/K30R/34R-GLP-2(1-34);
A2G/A18K(N^{ε}-PAO)propionyl)/K30R/34R-GLP-2(1-34);
A2G/D21K(N^{ε}-PAO)/K30R/34R-GLP-2(1-34);
A2G/N24K(N^{ε}-PAO)/K30R/34R-GLP-2(1-34);
A2G/Q28K(N^{ε}-PAO)/K30R/34R-GLP-2(1-34);
A2G/D3E/S5K(N^{ε}-PAO)/K30R/D33E/34R-GLP-2(1-34);
A2G/D3E/S7K(3-(N^{ε}-PAO)/K30R/D33E/34R-GLP-2(1-34);
A2G/D3E/D8K(N^{ε}-PAO)/K30R/D33E/34R-GLP-2(1-34);
A2G/D3E/E9K(N^{ε}-PAO)/K30R/D33E/34R-GLP-2(1-34);
A2G/D3E/M10K(N^{ε}-PAO)/K30R/D33E/34R-GLP-2(1-34);
A2G/D3E/N11K(N^{ε}-PAO)/K30R/D33E/34R-GLP-2(1-34);
A2G/D3E/T12K(N^{ε}-PAO)/K30R/D33E/34R-GLP-2(1-34);
A2G/D3E/I13K(N^{ε}-PAO)/K30R/D33E/34R-GLP-2(1-34);
A2G/D3E/L14K(N^{ε}-PAO)/K30R/D33E/34R-GLP-2(1-34);
A2G/D3E/D15K(N^{ε}-PAO)/K30R/D33E/34R-GLP-2(1-34);
A2G/D3E/N16K(N^{ε}-PAO)/K30R/D33E/34R-GLP-2(1-34);
A2G/D3E/L17K(N^{ε}-PAO)/K30R/D33E/34R-GLP-2(1-34);
A2G/D3E/A18K(N^{ε}-PAO)/K30R/D33E/34R-GLP-2(1-34);
A2G/D3E/D21K(N^{ε}-PAO)/K30R/D33E/34R-GLP-2(1-34);
A2G/D3E/N24K(N^{ε}-PAO)/K30R/D33E/34R-GLP-2(1-34); and
A2G/D3E/Q28K(N^{ε}-PAO)/K30R/D33E/34R-GLP-2(1-34).

In one embodiment of the invention the GLP-2 derivative is selected from the group consisting of
A2G/S5K(3-(PEG-amino)propionyl)/34R-GLP-2(1-34);
A2G/S7K(3-(PEG-amino)propionyl)/34R-GLP-2(1-34);
A2G/D8K(3-(PEG-amino)propionyl)/34R-GLP-2(1-34);
A2G/E9K(3-(PEG-amino)propionyl)/34R-GLP-2(1-34);
A2G/M10K(3-(PEG-amino)propionyl)/34R-GLP-2(1-34);
A2G/N11K(3-(PEG-amino)propionyl)/34R-GLP-2(1-34);
A2G/T12K(3-(PEG-amino)propionyl)/34R-GLP-2(1-34);
A2G/I13K(3-(PEG-amino)propionyl)/34R-GLP-2(1-34);
A2G/L14K(3-(PEG-amino)propionyl)/34R-GLP-2(1-34);
A2G/D15K(3-(PEG-amino)propionyl)/34R-GLP-2(1-34);
A2G/N16K(3-(PEG-amino)propionyl)/34R-GLP-2(1-34);
A2G/L17K(3-(PEG-amino)propionyl)/34R-GLP-2(1-34);
A2G/L17K((S)-4-carboxy-4-(PEG-amino)butanoyl)/34R-GLP-2(1-34);
A2G/L17K(4-(PEG-amino)butanoyl)/34R-GLP-2(1-34);
A2G/A18K(3-(PEG-amino)propionyl)/34R-GLP-2(1-34);
A2G/D21K(3-(PEG-amino)propionyl)/34R-GLP-2(1-34);
A2G/N24K(3-(PEG-amino)propionyl)/34R-GLP-2(1-34);
A2G/Q28K(3-(PEG-amino)propionyl)/34R-GLP-2(1-34);
A2G/S5K(3-(PEG-amino)propionyl)/K30R/34R-GLP-2(1-34);
A2G/S7K(3-(PEG-amino)propionyl)/K30R/34R-GLP-2(1-34);
A2G/D8K(3-(PEG-amino)propionyl)/K30R/34R-GLP-2(1-34);
A2G/E9K(3-(PEG-amino)propionyl)/K30R/34R-GLP-2(1-34);
A2G/M10K(3-(PEG-amino)propionyl)/K30R/34R-GLP-2(1-34);
A2G/N11K(3-(PEG-amino)propionyl)/K30R/34R-GLP-2(1-34);
A2G/T12K(3-(PEG-amino)propionyl)/K30R/34R-GLP-2(1-34);
A2G/I13K(3-(PEG-amino)propionyl)/K30R/34R-GLP-2(1-34);
A2G/L14K(3-(PEG-amino)propionyl)/K30R/34R-GLP-2(1-34);
A2G/D15K(3-(PEG-amino)propionyl)/K30R/34R-GLP-2(1-34);
A2G/N16K(3-(PEG-amino)propionyl)/K30R/34R-GLP-2(1-34);
A2G/L17K(3-(PEG-amino)propionyl)/K30R/34R-GLP-2(1-34);
A2G/L17K((S)-4-carboxy-4-(PEG-amino)butanoyl)/K30R/34R-GLP-2(1-34);
A2G/L17K(4-(PEG-amino)butanoyl)/K30R/34R-GLP-2(1-34);
A2G/A18K(3-(PEG-amino)propionyl)/K30R/34R-GLP-2(1-34);
A2G/D21K(3-(PEG-amino)propionyl)/K30R/34R-GLP-2(1-34);
A2G/N24K(3-(PEG-amino)propionyl)/K30R/34R-GLP-2(1-34);
A2G/Q28K(3-(PEG-amino)propionyl)/K30R/34R-GLP-2(1-34);
A2G/D3E/S5K(3-(PEG-amino)propionyl)/K30R/D33E/34R-GLP-2(1-34);
A2G/D3E/S7K(3-(PEG-amino)propionyl)/K30R/D33E/34R-GLP-2(1-34);
A2G/D3E/D8K(3-(PEG-amino)propionyl)/K30R/D33E/34R-GLP-2(1-34);
A2G/D3E/E9K(3-(PEG-amino)propionyl)/K30R/D33E/34R-GLP-2(1-34);
A2G/D3E/M10K(3-(PEG-amino)propionyl)/K30R/D33E/34R-GLP-2(1-34);
A2G/D3E/N11K(3-(PEG-amino)propionyl)/K30R/D33E/34R-GLP-2(1-34);
A2G/D3E/T12K(3-(PEG-amino)propionyl)/K30R/D33E/34R-GLP-2(1-34);
A2G/D3E/I13K(3-(PEG-amino)propionyl)/K30R/D33E/34R-GLP-2(1-34);
A2G/D3E/L14K(3-(PEG-amino)propionyl)/K30R/D33E/34R-GLP-2(1-34);
A2G/D3E/D15K(3-(PEG-amino)propionyl)/K30R/D33E/34R-GLP-2(1-34);
A2G/D3E/N16K(3-(PEG-amino)propionyl)/K30R/D33E/34R-GLP-2(1-34);
A2G/D3E/L17K(3-(PEG-amino)propionyl)/K30R/D33E/34R-GLP-2(1-34);
A2G/D3E/L17K((S)-4-carboxy-4-(PEG-amino)butanoyl)/K30R¢D33E/34R-GLP-2(1-34);
A2G/D3E/L17K(4-(PEG-amino)butanoyl)/K30R/D33E/34R-GLP-2(1-34);
A2G/D3E/A18K(3-(PEG-amino)propionyl)/K30R/D33E/34R-GLP-2(1-34);
A2G/D3E/D21K(3-(PEG-amino)propionyl)/K30R/D33E/34R-GLP-2(1-34);
A2G/D3E/N24K(3-(PEG-amino)propionyl)/K30R/D33E/34R-GLP-2(1-34);
A2G/D3E/Q28K(3-(PEG-amino)propionyl)/K30R/D33E/34R-GLP-2(1-34);
A2G/S5K(N^{ε}-PEG)/34R-GLP-2(1-34);
A2G/S7K(N^{ε}-PEG)/34R-GLP-2(1-34);
A2G/D8K(N^{ε}-PEG)/34R-GLP-2(1-34);
A2G/E9K(N^{ε}-PEG)/34R-GLP-2(1-34);
A2G/M10K(N^{ε}-PEG)/34R-GLP-2(1-34);
A2G/N11K(N^{ε}-PEG)/34R-GLP-2(1-34);
A2G/T12K(N^{ε}-PEG)/34R-GLP-2(1-34);
A2G/I13K(N^{ε}-PEG)/34R-GLP-2(1-34);
A2G/L14K(N^{ε}-PEG)/34R-GLP-2(1-34);
A2G/D15K(N^{ε}-PEG)/34R-GLP-2(1-34);
A2G/N16K(N^{ε}-PEG)/34R-GLP-2(1-34);
A2G/L17K(N^{ε}-PEG)/34R-GLP-2(1-34);
A2G/A18K(N^{ε}-PEG)/34R-GLP-2(1-34);
A2G/D21K(N^{ε}-PEG)/34R-GLP-2(1-34);
A2G/N24K(N^{ε}-PEG)/34R-GLP-2(1-34);
A2G/Q28K(N^{ε}-PEG)/34R-GLP-2(1-34);
A2G/S5K(N^{ε}-PEG)/K30R/34R-GLP-2(1-34);
A2G/S7K(N^{ε}-PEG)/K30R/34R-GLP-2(1-34);
A2G/D8K(N^{ε}-PEG)/K30R/34R-GLP-2(1-34);
A2G/E9K(N^{ε}-PEG)/K30R/34R-GLP-2(1-34);
A2G/M10K(N^{ε}-PEG)/K30R/34R-GLP-2(1-34);
A2G/N11K(N^{ε}-PEG)/K30R/34R-GLP-2(1-34);
A2G/T12K(N^{ε}-PEG)/K30R/34R-GLP-2(1-34);
A2G/I13K(N^{ε}-PEG)/K30R/34R-GLP-2(1-34);
A2G/L14K(N^{ε}-PEG)/K30R/34R-GLP-2(1-34);
A2G/D15K(N^{ε}-PEG)/K30R/34R-GLP-2(1-34);
A2G/N16K(N^{ε}-PEG)/K30R/34R-GLP-2(1-34);
A2G/L17K(N^{ε}-PEG)/K30R/34R-GLP-2(1-34);
A2G/A18K(N^{ε}-PEG)propionyl)/K30R/34R-GLP-2(1-34);
A2G/D21K(N^{ε}-PEG)/K30R/34R-GLP-2(1-34);
A2G/N24K(N^{ε}-PEG)/K30R/34R-GLP-2(1-34);
A2G/Q28K(N^{ε}-PEG)/K30R/34R-GLP-2(1-34);
A2G/D3E/S5K(N^{ε}-PEG)/K30R/D33E/34R-GLP-2(1-34);
A2G/D3E/S7K(3-(N^{ε}-PEG)/K30R/D33E/34R-GLP-2(1-34);
A2G/D3E/D8K(N^{ε}-PEG)/K30R/D33E/34R-GLP-2(1-34);
A2G/D3E/E9K(N^{ε}-PEG)/K30R/D33E/34R-GLP-2(1-34);
A2G/D3E/M10K(N^{ε}-PEG)/K30R/D33E/34R-GLP-2(1-34);
A2G/D3E/N11K(N^{ε}-PEG)/K30R/D33E/34R-GLP-2(1-34);
A2G/D3EFT12K(N^{ε}-PEG)/K30R/D33E/34R-GLP-2(1-34);
A2G/D3E/I13K(N^{ε}-PEG)/K30R/D33E/34R-GLP-2(1-34);
A2G/D3E/L14K(N^{ε}-PEG)/K30R/D33E/34R-GLP-2(1-34);
A2G/D3E/D15K(N^{ε}-PEG)/K30R/D33E/34R-GLP-2(1-34);
A2G/D3E/N16K(N^{ε}-PEG)/K30R/D33E/34R-GLP-2(1-34);
A2G/D3E/L17K(N^{ε}-PEG)/K30R/D33E/34R-GLP-2(1-34);
A2G/D3E/A18K(N^{ε}-PEG)/K30R/D33E/34R-GLP-2(1-34);
A2G/D3E/D21K(N^{ε}-PEG)/K30R/D33E/34R-GLP-2(1-34);
A2G/D3E/N24K(N^{ε}-PEG)/K30R/D33E/34R-GLP-2(1-34); and
A2G/D3E/Q28K(N^{ε}-PEG)/K30R/D33E/34R-GLP-2(1-34), wherein the PEG substituent in any of the compounds has a molecular weight from about 200 to about 100.000 Daltons.

In one embodiment the hydrophilic group is a sugar moiety.

In one embodiment the hydrophilic substituent comprises an 2-acetamido-2-deoxy-β-D-glucopyranosyl moiety.

In one embodiment of the invention the GLP-2 derivative is selected from the group consisting of D3E/N16N(2-acetamido-2-deoxy-β-D-glucopyranosyl)/K30R-GLP-2(1-33)) and D3E/M10L/N11N(2-acetamido-2-deoxy-p-D-glucopyranosyl)-GLP-2(1-33).

In a further embodiment, the present invention relates to a GLP-2 derivative in which the C-terminal amino acid residue is present in the form of the amide.

The parent GLP-2 peptide can be produced by a method which comprises culturing a host cell containing a DNA sequence encoding the GLP-2 peptide and capable of expressing the GLP-2 peptide in a suitable nutrient medium under conditions permitting the expression of the GLP-2 peptide, after which the resulting GLP-2 peptide is recovered from the culture.

The medium used, to culture the cells may be any conventional medium suitable for growing the host cells, such as minimal or complex media containing appropriate supplements. Suitable media are available from commercial suppliers or may be prepared according to published recipes (e.g. in catalogues of the American Type Culture Collection). The GLP-2 peptide produced by the cells may then be recovered from the culture medium by conventional procedures including separating the host cells from the medium by centrifugation or filtration, precipitating the proteinaceous components of the supernatant or filtrate by means of a salt, e.g. ammonium sulphate, purification by a variety of chromatographic procedures, e.g. ion exchange chromatography, gelfiltration chromatography, affinity chromatography, or the like, dependent on the type of GLP-2 peptide in question.

The DNA sequence encoding the parent GLP-2 peptide may suitably be of genomic or cDNA origin, for instance obtained by preparing a genomic or cDNA library and screening for DNA sequences coding for all or part of the GLP-2 peptide by hybridisation using synthetic oligonucleotide probes in accordance with standard techniques (see, for example, Sambrook, J, Fritsch, E F and Maniatis, T, Molecular Cloning: A Laboratory Manual, Cold Spring Harbor Laboratory Press, New York, 1989). The DNA sequence encoding the GLP-2 peptide may also be prepared synthetically by established standard methods, e.g. the phosphoamidite method described by Beaucage and Caruthers, Tetrahedron Letters 22 (1981), 1859-1869, or the method described by Matthes et al., EMBO Journal 3 (1984), 801-805. The DNA sequence may also be prepared by polymerase chain reaction using specific primers, for instance as described in U.S. Pat. No. 4,683,202 or Saiki et al., Science 239 (1988), 487-491.

The DNA sequence may be inserted into any vector which may conveniently be subjected to recombinant DNA procedures, and the choice of vector will often depend on the host cell into which it is to be introduced. Thus, the vector may be an autonomously replicating vector, i.e. a vector which exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated.

The vector is preferably an expression vector in which the DNA sequence encoding the GLP-2 peptide is operably linked to additional segments required for transcription of the DNA, such as a promoter. The promoter may be any DNA sequence which shows transcriptional activity in the host cell of choice and may be derived from genes encoding proteins either homologous or heterologous to the host cell. Examples of suitable promoters for directing the transcription of the DNA encoding the GLP-2 peptide of the invention in a variety of host cells are well known in the art, cf. for instance Sambrook et al., supra.

The DNA sequence encoding the GLP-2 peptide may also, if necessary, be operably connected to a suitable terminator, polyadenylation signals, transcriptional enhancer sequences, and translational enhancer sequences. The recombinant vector of the invention may further comprise a DNA sequence enabling the vector to replicate in the host cell in question.

The vector may also comprise a selectable marker, e.g. a gene the product of which complements a defect in the host cell or one which confers resistance to a drug, e.g. ampicillin, kanamycin, tetracyclin, chloramphenicol, neomycin, hygromycin or methotrexate.

To direct a parent GLP-2 peptide of the present invention into the secretory pathway of the host cells, a secretory signal sequence (also known as a leader sequence, prepro sequence or pre sequence) may be provided in the recombinant vector. The secretory signal sequence is joined to the DNA sequence encoding the GLP-2 peptide in the correct reading frame. Secretory signal sequences are commonly positioned 5′ to the DNA sequence encoding the GLP-2 peptide. The secretory signal sequence may be that normally associated with the GLP-2 peptide or may be from a gene encoding another secreted protein.

The procedures used to ligate the DNA sequences coding for the present GLP-2 peptides, the promoter and optionally the terminator and/or secretory signal sequence, respectively, and to insert them into suitable vectors containing the information necessary for replication, are well known to persons skilled in the art (cf., for instance, Sambrook et al., supra).

The host cell into which the DNA sequence or the recombinant vector is introduced may be any cell which is capable of producing the present GLP-2 peptides and includes bacteria, yeast, fungi and higher eukaryotic cells. Examples of suitable host cells well known and used in the art are, without limitation, E. coli, Saccharomyces cerevisiae, or mammalian BHK or CHO cell lines.

The parent GLP-2 peptide can also be produced using standard methods of solid-phase peptide synthesis techniques. Peptide synthesizers are commercially available from for example Applied Biosystems in Foster City Calif. Reagents for solid phase synthesis are commercially available from, for example Midwest Biotech (Fishers, In). Solid phase peptide synthesizers can be used according to manufactures instruction for blocking interfering groups, protecting the amino acid to be reacted, coupling, decoupling, and capping of unreacted amino acids.

Typically an α-N-carbomoyl protected amino acid and an N-terminal amino acid on the growing peptide chain on a resin is coupled at room temperature in an inert solvent such as dimethylformamide. N-methylpyrrolidone or methylenechloride in the present of a coupling reagent such as dicyclohexylcarbodiimide and 1-hydroxybenzotriazole and a base such as diisopropylethylamine. The α-N-carbomoyl protecting group is removed from the resulting peptide resin using a reagent such as trifluoroacetic acid or piperidine, and the coupling is repeated with the next desired N-protected amino acid to be added to the peptide chain. Suitable amine protecting groups are well known in the art and are described, for example in Green and Wuts, “Protecting Groups in Organic Synthesis”, John Wiley and Sons, 1999, the entire teaching of which are incorporated by reference. Examples include t-butyloxycarbonyl (tBoc) and fluorenylmethoxycarbonyl (Fmoc).

The peptides can be synthesized using standard automated solid-phase synthesis protocols using t-butyloxycarbonyl- or fluorenylmethoxycarbonyl-alpha-amino acids with appropriate side-chain protection.

The GLP-2 derivatives of the invention can be prepared by introducing the Polymeric Compounds into the parent GLP-2 peptide using standard methods. Pegylation of peptide and proteins is a well established technique see for example (Roberts et al. Adv. Drug Delivery Revl. 54: 459-476 (2002)., the contents of which is hereby incorporated in its entirety by reference.

N^{ε}-acylation of a Lys residue can be carried out by using an activated amide of the acyl group to be introduced as the acylating agent, e.g. the amide with benzotriazole. The acylation is carried out in a polar solvent in the presence of a base.

Pharmaceutical Compositions Pharmaceutical compositions containing a GLP-2 derivative according to the present invention may be administered parenterally to patients in need of such a treatment. Parenteral administration may be performed by subcutaneous, intramuscular or intravenous injection by means of a syringe, optionally a pen-like syringe. Alternatively, parenteral administration can be performed by means of an infusion pump. A further option is a composition which may be a powder or a liquid for the administration of the GLP-2 derivative in the form of a nasal or pulmonal spray. As a still further option, the GLP-2 derivatives of the invention can also be administered transdermally, e.g. from a patch, optionally a iontophoretic patch, or transmucosally, e.g. bucally.

Pharmaceutical compositions containing a GLP-2 derivative of the present invention may be prepared by conventional techniques, e.g. as described in Remington's Pharmaceutical Sciences, 1985 or in Remington: The Science and Practice of Pharmacy, 19^{th} edition, 1995.

Thus, the injectable compositions of the GLP-2 derivative of the invention can be prepared using the conventional techniques of the pharmaceutical industry which involves dissolving and mixing the ingredients as appropriate to give the desired end product.

Thus, according to one procedure, the GLP-2 derivative is dissolved in an amount of water which is somewhat less than the final volume of the composition to be prepared. An isotonic agent, a preservative and a buffer is added as required and the pH value of the solution is adjusted—if necessary—using an acid, e.g. hydrochloric acid, or a base, e.g. aqueous sodium hydroxide as needed. Finally, the volume of the solution is adjusted with water to give the desired concentration of the ingredients.

Examples of isotonic agents are sodium chloride, mannitol and glycerol.

Examples of preservatives are phenol, m-cresol, methyl p-hydroxybenzoate and benzyl alcohol.

Examples of suitable buffers are sodium acetate and sodium phosphate.

Further to the above-mentioned components, solutions containing a GLP-2 derivative according to the present invention may also contain a surfactant in order to improve the solubility and/or the stability of the derivative.

A composition for nasal administration of GLP-2 may, for example, be prepared as described in European Patent No. 272097 (to Novo Nordisk A/S) or in WO 93/18785.

The GLP-2 derivatives of this invention can be used in the treatment of various diseases. The particular GLP-2 derivative to be used and the optimal dose level for any patient will depend on the disease to be treated and on a variety of factors including the efficacy of the specific peptide derivative employed, the age, body weight, physical activity, and diet of the patient, on a possible combination with other drugs, and on the severity of the case. It is recommended that the dosage of the GLP-2 derivative of this invention be determined for each individual patient by those skilled in the art in a similar way as for known parent GLP-2 peptides.

The pharmacological properties of the compounds of the invention can be tested e.g. as described in our Intemational Patent Application No. PCT/DK97/00086 the contents of which is hereby incorporated in its entirety by reference.

In the present context the three-letter or one-letter indications of the amino acids have been used in their conventional meaning as indicated in table 1. Unless indicated explicitly, the amino acids mentioned herein are L-amino acids. Further, the left and right ends of an amino acid sequence of a peptide are, respectively, the N- and C-termini unless otherwise specified.

TABLE 1
Abbreviations for amino acids:

| Amino acid | Tree-letter code | One-letter code |
|---|---|---|
| Glycine | Gly | G |
| Proline | Pro | P |
| Alanine | Ala | A |
| Valine | Val | V |
| Leucine | Leu | L |
| Isoleucine | Ile | I |
| Methionine | Met | M |
| Cysteine | Cys | C |
| Phenylalanine | Phe | F |
| Tyrosine | Tyr | Y |
| Tryptophan | Trp | W |
| Histidine | His | H |
| Lysine | Lys | K |
| Arginine | Arg | R |
| Glutamine | Gln | Q |
| Asparagine | Asn | N |
| Glutamic Acid | Glu | E |
| Aspartic Acid | Asp | D |
| Serine | Ser | S |
| Threonine | Thr | T |

A simple system is used herein to describe peptides, fragments, analogs and derivatives of GLP-2. Thus, for example, R20K-GLP-2(1-31) designates a fragment of GLP-2 formally derived from GLP-2 by deleting the amino acid residues at position 32 and 33 of SEQ ID NO:1 and substituting the naturally occurring amino acid residue arginine at position 20 of SEQ ID NO:1 by a lysine. Similarly, R20K(N^{ε}-PEG)/K30R-GLP-2(1-33) designates a derivative of a GLP-2 peptide analog formally derived from GLP-2 by exchange of the naturally occurring amino acid residue lysine in position 30 of SEQ ID NO:1 with an arginine residue and exchange of the naturally occurring amino acid residue arginine in position 20 of SEQ ID NO:1 with a lysine residue and pegylation of the E-amino group of the lysine residue in position 20 relative to the amino acid sequence of SEQ ID NO:1.

Similarly, L17K(3-(PEG-amino)propionyl)/K30R-GLP-2(1-33) designates a derivative of a GLP-2 peptide analog formally derived from GLP-2 by exchange of the naturally occurring amino acid residue lysine in position 30 of SEQ ID NO:1 with an arginine residue and exchange of the naturally occurring amino acid residue leucine in position 17 of SEQ ID NO:1 with a lysine residue and pegylation of the E-amino group of the lysine residue in position 17 relative to the amino acid sequence of formula I by means of the spacer P-alanine. Several changes in the sequence are separated in the notation by a slash (“/”).
Derivatized amino acids are noted in a way, in which the moiety it is derivatized with is described in brackets following the one-letter code of the respective amino acid. The specific positions of the derivatization of each of the possible amino acids are defined as shown.

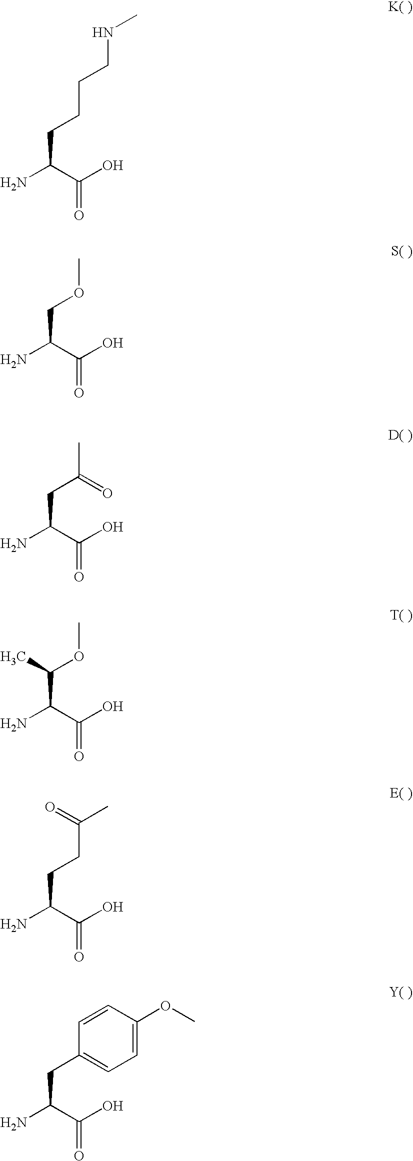

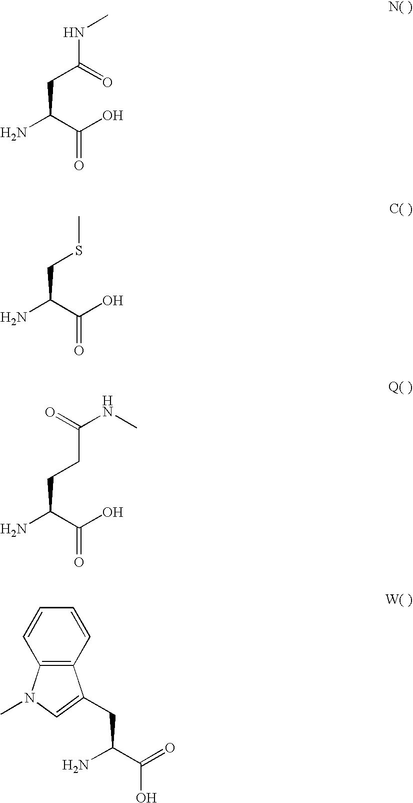

For the following amino acids, both of two possible attachment-points are included into the definition, unless stated otherwise.

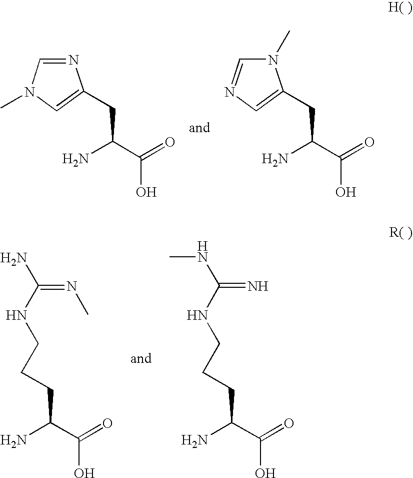

The present invention is further illustrated by the following examples which, however, are not to be construed as limiting the scope of protection. The features disclosed in the foregoing description and in the following examples may, both separately and in any combination thereof, be material for realising the invention in diverse forms thereof.

### BRIEF DESCRIPTION OF THE DRAWINGS

FIG. 1 The amino acid sequence of the 33 residues human GLP-2. The N-terminal His-Ala indicates the sequence cleaved of aminopeptidase dipeptidyl peptidase IV during metabolism of GLP-2. The Arg2O and Lys3O residues are the two basic amino acid residues in GLP-2.

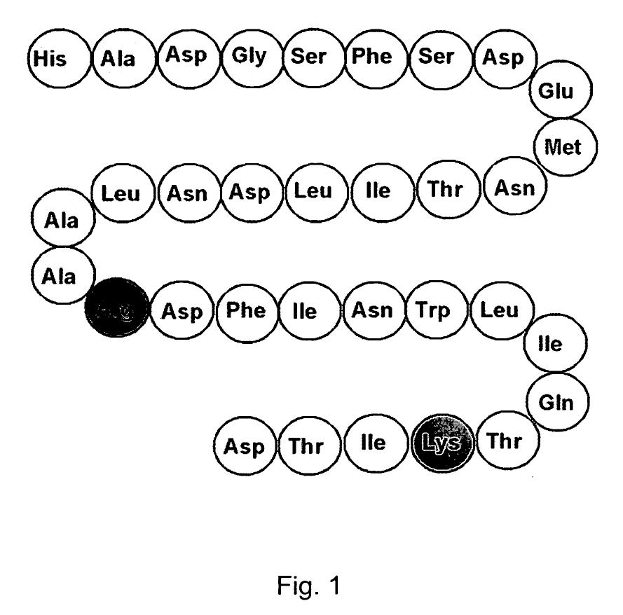

FIG. 2 List of amino acid sequences used in the detailed description of the invention.

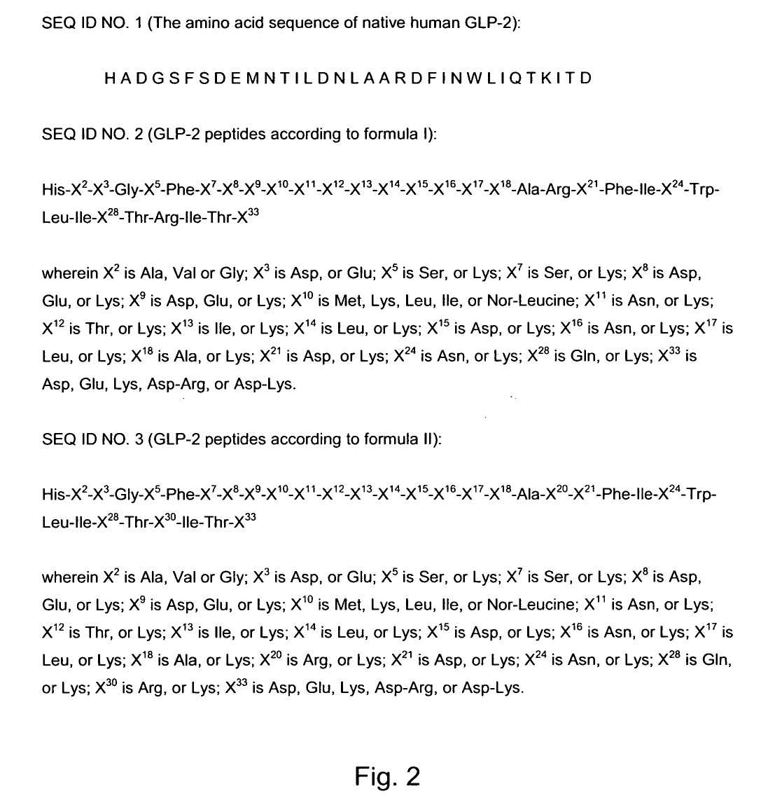

### ABBREVIATIONS

r.t. retention time
TFE trifluoroethanol
DIEA diisopropylethylamine
CH₃CN acetonitrile
DMF N,N dimethylformamide
HBTU 2-(1H-Benzotriazol-1-yl-)-1,1,3,3 tetramethyluronium hexafluorophosphate
Fmoc 9 H-fluoren-9-ylmethoxycarbonyl
Boc tert butyloxycarbonyl
OtBu tert butyl ester
tBu tert butyl
Trt triphenylmethyl
Pmc 2,2,5,7,8-Pentamethyl-chroman-6-sulfonyl
Dde 1-(4,4-Dimethyl-2,6-dioxocyclohexylidene)ethyl
DCM dichloromethane
TIS triisopropylsilane
TFA: trifluoroacetic acid
Et₂O: diethyl ether
NMP 1-Methylpyrrolidin-2-one
Fmoc-(FmocHmb)Gly-OH N-α-Fmoc-α-(2-Fmoc-oxy-4-methoxybenzyl)-glycine
   HBTU

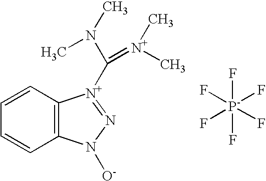

HOBT: 1-Hydroxybenzotriazole

HATU:N-[(Dimethylamino-1H-1,2,3 triazolo[4,5-b]pyridine-1-ylmethylene]-N-methylmethanaminium hexafluorophosphate N-oxide

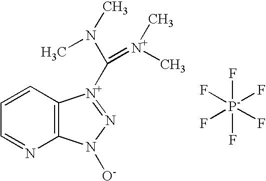

HOAT: 1-Hydroxy-7azabenzotriazole

TFA: trifluoroacetic acid

HPLC-Methods:

Method A1

The RP-analysis was performed using a Waters 2690 systems fitted with a Waters 996 diode array detector. UV detections were collected at 214, 254, 276, and 301 nm on a 218TP54 4.6 mm×250 mm 5μ C-18 silica column (The Seperations Group, Hesperia), which was eluted at 1 ml/min at 42° C. The column was equilibrated with 10% of a 0,5 M ammonium sulfate, which was adjusted to pH 2.5 with 4M sulfuric acid. After injection, the sample was eluted by a gradient of 0% to 60% acetonitrile in the same aqueous buffer during 50 min.

Method B1

The Reversed Phase-analysis was performed using a Waters 2690 systems fitted with a Waters 996 diode array detector. UV detections were collected at 214, 254, 276, and 301 nm on a 218TP54 4.6 mm×250 mm 5μ C-18 silica column (The Seperations Group, Hesperia), which was eluted at 0,5 ml/min at 42° C. The column was equilibrated with an aqueous solution of TFA in water (0.1%). After injection, the sample was eluted by a gradient of 0% to 60% acetonitrile (+0.1% TFA) in an aqueous solution of TFA in water (0.1%) during 50 min.

Method B6

The Reversed Phase-analysis was performed using a Waters 2690 systems fitted with a Waters 996 diode array detector. UV detections were collected at 214, 254, 276, and 301 nm on a 218TP54 4.6 mm×250 mm 5μ C-18 silica column (The Seperations Group, Hesperia), which was eluted at 0,5 ml/min at 42° C. The column was equilibrated with an aqueous solution of TFA in water (0.1%). After injection, the sample was eluted by a gradient of 0% to 90% acetonitrile (+0.1% TFA) in an aqueous solution of TFA in water (0.1%) during 50 min.

Method 02-A1

HPLC (Method 02-A1): The RP-analyses was performed using a Alliance Waters 2695 system fitted with a Waters 2487 dualband detector. UV detections were collected using a Symmetry C18 , 3.5 um, 3.0 mm×100 mm column. The elution was performed with a linear gradient of 0-60% of a 0.5% solution of diammonium sulfate in water and 100-40% of water over 15 minutes at a flow-rate of 0.75 ml/min at a temperature of 42° C.

Method 02-B4

HPLC (Method 02-B4): The RP-analyses was performed using a Alliance Waters 2695 system fitted with a Waters 2487 dualband detector. UV detections were collected using a Symmetry C18 , 3.5 um, 3.0 mm×100 mm column. The elution was performed with a linear gradient of 5-90% of a 0.1% solution of trifluoroacetic acid acetonitrile and 95-10% of a 0.1 solution of trifluoroacetic acid in water over 15 minutes at a flow-rate of 1.0 ml/min at a temperature of 42° C.

### EXAMPLE 1

### L17K(mPEGpropionyl)/K30R-GLP-2(1-33),wherein mPEG has a molecular weight of approximately 2 kDa

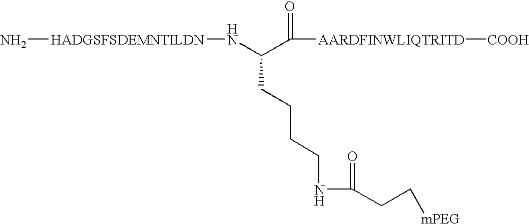 1.a Synthesis of the protected peptidyl resin.

Boc-His(Boc)-Ala-Asp(OtBu)-Gly(Hmb)-Ser(tBu)-Phe-Ser(tBu)-Asp(OtBu)-Glu(OtBu)-Met-Asn(Trt) -Thr(tBu)-Ile-Leu-Asp(OtBu)-Asn(Trt)-Lys(Dde)-Ala-Ala-Arg(Pmc)-Asp(OtBu)-Phe-Ile-Asn(Trt) -Trp(Boc)-Leu-Ile-Gln(Trt)-Thr(tBu)-Arg(Pmc)-Ile-Thr(tBu)-Asp(OtBu)-Wang resin was performed according to the Fmoc strategy on an Applied Biosystems 433A peptide synthesizer in 0.25 mmol scale using the manufacturer supplied FastMoc UV protocols which employ HBTU or HATU mediated couplings in NMP, and UV monitoring of the deprotection of the Fmoc protection group. The starting resin (438 mg) used for the synthesis was Fmoc-Asp(OtBu)-Wang resin (Merck Biosciences GmbH, Germany. cat. #: 04-12-2047) with a substitution capacity of 0.57 mmol/g. The protected amino acid derivatives used were (2S)-6-[1-(4,4-Dimethyl-2,6-dioxo-cyclohexylidene)-ethylamino]-2-(9H-fluoren-9-ylmethoxycarbonylamino)-hexanoic acid (Fmoc-Lys(Dde)-OH), Fmoc-Arg(Pmc)-OH, Fmoc-Leu-OH, Fmoc-Trp(Boc)-OH, Fmoc-Ala-OH, Fmoc-Ile-OH, Fmoc-Phe-OH, Fmoc-Glu(OtBu)-OH, Fmoc-Gln(Trt)-OH, Fmoc-Asn(Trt)-OH, Fmoc-Ser(tBu)-OH, Fmoc-Asp(OtBu)-OH, Fmoc-Thr(tBu)-OH, Fmoc-Met-OH, Boc-His(Boc)-OH and Fmoc-(FmocHmb)Gly-OH.

1.b Deprotection of Dde

The protected peptidyl resin resulting from (1.a) (300 mg) was washed in NMP:DCM1:1 (15 ml) twice. A freshly prepared solution of hydrazine hydrate 2% in NMP (12ml) was added. The reaction mixture was shaken for 5 min at room temperature, and then filtered. The hydrazine treatment was repeated with hydrazine hydrate 2% in NMP (20 ml) for 15 min. After this the resin was washed extensively with NMP, DCM and NMP.

1.c Pegylation

The Dde deprotected resin was suspended in NMP (20 ml). mPEG-Succinimidyl propionate (mPEG-SPA) (Nektar Therapeutics, CA, USA, cat. #: 2M4MOD0147 ) (6 g, 0.3 mmol.) was added and the suspension was shaken overnight. Then the resin was isolated by filtration and washed extensively with NMP, DCM, 2-propanol, methanol and Et₂O and dried in vacuo.

1.d Cleavage of the Product

The resin from 1.c was stirred for 3 h at room temperature with a mixture of 500 μl TIS, 500 μl H₂O and 20 ml TFA. The resin was removed by filtration and washed with 3 ml TFA. The collected filtrates were concentrated in vacuo to 5 ml and the crude product was precipitated by addition of 30 ml Et₂O followed by centrifugation. The pellet was washed with 40 ml Et₂O two times and then air dried.

1.e Purification of Product.

The crude peptide was dissolved in H₂O/NH₃ (99:1) (10 ml) and purified by preparative HPLC in 2 runs on a 5 mm×250 mm column packed with C-18 silica. The column was eluted with a gradient of CH₃CN from 28 to 48% against 0.1% TFA/H₂O at 20 ml/min at room temperature for 40 min. The peptide containing fractions were collected, diluted with 3 volumes of H₂O and lyophilized. The final product obtained was characterized by HPLC.

| Analytical method | Result |
|---|---|
| HPLC B6 | r.t.: 28, 43 min., |

### EXAMPLE 2

### D3E/N16N(2-acetamido-2-deoxy-β-D-glucopyranosyl)/K30R-GLP-2(1-33)

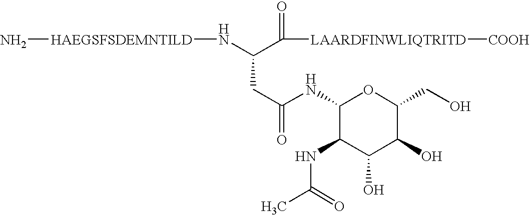

The peptide D3E/N16N(2-acetamido-2-deoxy-3,4,6-tri-O-acetyl-β-D-glucopyranosyl)/K30R-GLP-2(1-33) was prepared on a Applied Biosystems 433A Peptide Synthesizer, on a Wang-resin (loading 1.07 mmol/g) applying a standard FMOC strategy using HBTU/HOBT as coupling reagent. (S)-N^{γ}-(2-acetamido-2-deoxy-3,4,6-tri-O-acetyl-β-D-glucopyranosyl)-N^{α}-((9H-9-fluorenyl)methoxycarbonyl)asparagine (commercially available at e.g. Bachem) at position 16, FMOC-Asp(OtBu)-OH at position 15, and FMOC-Leu-OH at position 14 were coupled using HATU/HOAT. The peptide was cleaved from the resin and purified on HPLC.
HPLC (Method B4): 7.16 min, 7.29 min, and 7.47 min.
MALDI-TOF: [M]⁺: 4132.38, [M-Ac]⁺: 4089.89; [M-2 Ac]⁺: 4047.46.

The peptide was dissolved in a 5% solution of hydrazine in water (10 ml). This solution was stirred for 1 h. It was diluted with water (10 ml) and purified on a HPLC, using a gradient of 30-60% acetonitrile in water in a 0.1% buffer of TFA. The yield of 1.2 mg was determined by UV absorption at 214 nm assuming an absorption coefficient of 1.7×10⁶ 1×g⁻¹×cm⁻¹.

HPLC: (Method Al): 36.46 min

HPLC: (Method B1): 38.12 min

MS: 1337 [M³⁺]; 1004 [M⁴⁺+1], 803 [M⁵⁺+1].

### EXAMPLE 3

### D3E/M10L/N11N(2-acetamido-2-deoxy-β-D-glucopyranosyl)-GLP-2(1-33)

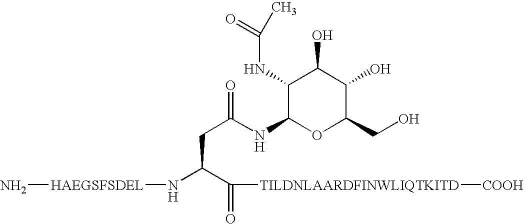

The title compound was prepared as described for D3E/N16N(2-acetamido-2-deoxy-β-D-glucopyranosyl)/K30R-GLP-2(1-33).

HPLC: (Method: 02-A1): 13.07 min.

HPLC: (Method: 02-B4): 8.70 min.

MS: 1323 [M³⁺+1], 992 [M⁴⁺+1], 794 [M⁵⁺+1], 662 [M⁶⁺+1].

1. A GLP-2 derivative comprising a GLP-2 peptide, wherein a hydrophilic substituent is attached to one or more amino acid residues at a position relative to the amino acid sequence of SEQ ID NO:1 independently selected from the list consisting of D3, S5, S7, D8, E9, M10, N11, T12, I13, L14, D15, N16, L17, A18, R20, D21, N24, Q28, and D33.

2. The GLP-2 derivative according to claim 1, wherein the GLP-2 peptide comprises the amino acid sequence of formula II
His-X²-X³-Gly-X⁵-Phe-X⁷-X⁸-X⁹-X¹⁰-X¹¹-X¹²-X¹³-X¹⁴-X¹⁵-X¹⁶-X¹⁷-X¹⁸-Ala-X²⁰-X²¹-Phe-Ile-X²⁴-Trp-Leu-Ile-X²⁸-Thr-X³⁰-Ile-Thr-X³³ (formula II)
or a fragment thereof; wherein X² is Ala, Val or Gly; X³ is Asp, or Glu; X⁵ is Ser, or Lys; X⁷ is Ser, or Lys; X⁸ is Asp, Glu, or Lys; X⁹ is Asp, Glu, or Lys; X¹⁰ is Met, Lys, Leu, Ile, or Nor-Leucine; X¹¹ is Asn, or Lys; X¹² is Thr, or Lys; X¹³ is Ile, or Lys; X¹⁴ is Leu, or Lys; X¹⁵ is Asp, or Lys; X¹⁶ is Asn, or Lys; X¹⁷ is Leu, or Lys; X¹⁸ is Ala, or Lys; X²⁰ is Arg, or Lys; X²¹ is Asp, or Lys; X²⁴ is Asn, or Lys; X²⁸ is Gln, or Lys; X³⁰ is Arg, or Lys; X³³ is Asp, Glu, Lys, Asp-Arg, or Asp-Lys (formula II).

3. The GLP-2 derivative according to claim 1, wherein the GLP-2 peptide comprises the amino acid sequence of formula I
His-X²-X³-Gly-X⁵-Phe-X⁷-X⁸-X⁹-X¹⁰-X¹¹-X¹²-X¹³-X¹⁴-X¹⁵-X¹⁶-X¹⁷-X¹⁸-Ala-Arg-X²¹-Phe-Ile-X²⁴-Trp-Leu-Ile-X²⁸-Thr-Arg-Ile-X³³ (formula II)
or a fragment thereof; wherein X² is Ala, Val or Gly; X³ is Asp, or Glu; X⁵ is Ser, or Lys; X⁷ is Ser, or Lys; X⁸ is Asp, Glu, or Lys; X⁹ is Asp, Glu, or Lys; X¹⁰ is Met, Lys, Leu, Ile, or Nor-Leucine; X¹¹ is Asn, or Lys; X¹² is Thr, or Lys; X¹³ is Ile, or Lys; X¹⁴ is Leu, or Lys; X¹⁵ is Asp, or Lys; X¹⁶ is Asn, or Lys; X¹⁷ is Leu, or Lys; X¹⁸ is Ala, or Lys; X²⁰ is Arg, or Lys; X²¹ is Asp, or Lys; X²⁸ is Gln, or Lys; X³³ is Asp, Glu, Lys, Asp-Arg, or Asp-Lys.

4. The GLP-2 derivative according to claim 1, wherein the GLP-2 peptide consists of the amino acid sequence of formula I
His-X²-X³-Gly-X⁵-Phe-X⁷-X⁸-X⁹-X¹⁰-X¹¹-X¹²-X¹³-X¹⁴-X¹⁵-X¹⁶-X¹⁷-X¹⁸-Ala-Arg-X²¹-Phe-Ile-X²⁴-Trp-Leu-Ile-X²⁸-Thr-Arg-Ile-Thr-X³³ (formula II)
or a fragment thereof; wherein X² is Ala, Val or Gly; X³ is Asp, or Glu; X⁵ is Ser, or Lys; X⁷ is Ser, or Lys; X⁸ is Asp, Glu, or Lys; X⁹ is Asp, Glu, or Lys; X¹⁰ is Met, Lys, Leu, Ile, or Nor-Leucine; X¹¹ is Asn, or Lys; X¹² is Thr, or Lys; X¹³ is Ile, or Lys; X¹⁴ is Leu, or Lys; X¹⁵ is Asp, or Lys; X¹⁶ is Asn, or Lys; X¹⁷ is Leu, or Lys; X¹⁸ is Ala, or Lys; X²⁰ is Arg, or Lys; X²¹ is Asp, or Lys; X²⁴ is Asn, or Lys; X²⁸ is Gln, or Lys; X³³ is Asp, Glu, Lys, Asp-Arg, or Asp-Lys.

5. The GLP-2 derivative according to claim 2, wherein (a) X² is Ala or Gly; (b) X³ is Asp or Glu; (C) X⁵ is Ser; (d) X⁷ is Ser; (e) X⁸ is Asp or Glu; (f) X⁹ is Asp or Glu; (g) X¹⁰ is selected from the group consisting of Met, Leu, Ile, and Nor-Leucine; (h) X¹¹ is Asn; (i) X¹² is Thr; (j) X¹³ is Ile; (k) X¹⁴ is Leu; (l) X¹⁵ is Asp; (m) X¹⁶ is Asn; (n) X¹⁷ is Leu; (o) X¹⁸ is Ala; (p) X²¹ is Asp; (q) X²⁴ is Asn; (r) X²⁸ is Gln; (S) X³³ is Asp or Glu; or (t) the GLP-2 derivative is characterized by comprising a GLP-2 peptide characterized by any combination of (a)-(s).

6. The GLP-2 derivative of claim 2, wherein one or more amino acid independently selected from the list consisting of X⁵, X⁷, X⁸, X⁹, X¹⁰, X¹¹, X¹², X¹³, X¹⁴, X¹⁵, X¹⁶, X¹⁷, X¹⁸, X²⁰, X²¹, X²⁴, X²⁸, and X³³ is a Lys.

6. The GLP-2 derivative of claim 5, wherein one or more amino acid independently selected from the list consisting of X⁵, X⁷, X⁸, X⁹, X¹⁰, X¹¹, X¹², X¹³, X¹⁴, X¹⁵, X¹⁶, X¹⁷, X¹⁸, X²⁰, X²¹, X²⁴, X²⁸, and X³³ is a Lys.

7. The GLP-2 derivative according to claim 1, wherein the peptide is selected from the list consisting of
GLP-2(1-33);
A2G-GLP-2(1-33);
K30R-GLP-2(1-33);
S5K-GLP-2(1-33);
S7K-GLP-2(1-33);
D8K-GLP-2(1-33);
E9K-GLP-2(1-33);
M10K-GLP-2(1-33);
N11K-GLP-2(1-33);
T12K-GLP-2(1-33);
I13K-GLP-2(1-33);
L14K-GLP-2(1-33);
D15K-GLP-2(1-33);
N16K-GLP-2(1-33);
L17K-GLP-2(1-33);
A18K-GLP-2(1-33);
D21K-GLP-2(1-33);
N24K-GLP-2(1-33);
Q28K-GLP-2(1-33);
A2G/34R-GLP-2(1-34);
S5K/K30R-GLP-2(1-33);
S7K/K30R-GLP-2(1-33);
D8K/K30R-GLP-2(1-33);
E9K/K30R-GLP-2(1-33);
M10K/K30R-GLP-2(1-33);
N11K/K30R-GLP-2(1-33);
T12K/K30R-GLP-2(1-33);
I13K/K30R-GLP-2(1-33);
L14K/K30R-GLP-2(1-33);
D15K/K30R-GLP-2(1-33);
N16K/K30R-GLP-2(1-33);
L17K/K30R-GLP-2(1-33);
A18K/K30R-GLP-2(1-33);
D21K/K30R-GLP-2(1-33);
N24K/K30R-GLP-2(1-33);
Q28K/K30R-GLP-2(1-33);
K30R/D33K-GLP-2(1-33);
D3E/K30R/D33E-GLP-2(1-33);
D3E/S5K/K30R/D33E-GLP-2(1-33);
D3E/S7K/K30R/D33E-GLP-2(1-33);
D3E/D8K/K30R/D33E-GLP-2(1-33);
D3E/E9K/K30R/D33E-GLP-2(1-33);
D3E/M10K/K30R/D33E-GLP-2(1-33);
D3E/N11K/K30R/D33E-GLP-2(1-33);
D3E/T12K/K30R/D33E-GLP-2(1-33);
D3E/I13K/K30R/D33E-GLP-2(1-33);
D3E/L14K/K30R/D33E-GLP-2(1-33);
D3E/D15K/K30R/D33E-GLP-2(1-33);
D3E/N16K/K30R/D33E-GLP-2(1-33);
D3E/L17K/K30R/D33E-GLP-2(1-33);
D3E/A18K/K30R/D33E-GLP-2(1-33);
D3E/D21K/K30R/D33E-GLP-2(1-33);
D3E/N24K/K30R/D33E-GLP-2(1-33); and
D3E/Q28K/K30R/D33E-GLP-2(1-33).

8. The GLP-2 derivative according to claim 1, wherein said hydrophilic substituent comprises H(OCH₂CH₂)ₙO— wherein n>4 with a molecular weight from about 200 to about 100.000 daltons.

9. The GLP-2 derivative according to claim 1, wherein said hydrophilic substituent comprises CH₃O—(CH₂CH₂O)ₙ—CH₂CH₂—O— wherein n>4 with a molecular weight from about 200 to about 100.000 daltons.

10. The GLP-2 derivative according to claim 1, wherein said hydrophilic substituent is attached to an amino acid residue in such a way that a carboxyl group of the hydrophilic substituent forms an amide bond with an amino group of the amino acid residue.

11. The GLP-2 derivative according to claim 1, wherein said hydrophilic substituent is attached to an amino acid residue in such a way that a carboxy group of the hydrophilic substituent forms an carbamate bond with an amino group of the amino acid residue.

12. The GLP-2 derivative according to claim 1, wherein said hydrophilic substituent is attached to an amino acid residue in such a way that a alkyl group of the hydrophilic substituent forms an secondary amine bond with an amino group of the amino acid residue.

13. The GLP-2 derivative according to claim 1, wherein said hydrophilic substituent is attached to an amino acid residue in such a way that an amino group of the hydrophilic substituent forms an amide bond with a carboxyl group of the amino acid residue.

14. The GLP-2 derivative according to claim 1, wherein said hydrophilic substituent is attached to said GLP-2 peptide by means of a spacer that is selected from (a) unbranched alkane α,ω-dicarboxylic acid group having from 1 to 7 methylene groups, such as two methylene groups which spacer forms a bridge between an amino group of the GLP-2 peptide and an amino group of said hydrophilic substituent or (b) an amino acid residue except a Cys residue, or a dipeptide.

15. The GLP-2 derivative according to claim 14, wherein said spacer is selected from the list consisting of H-alanine, gamma-aminobutyric acid (GABA), γ-glutamic acid, Lys, Asp, Glu, a dipeptide containing Asp, a dipeptide containing Glu, or a dipeptide containing Lys.

16. A GLP-2 derivative according to claim 1, which is selected from the group consisting of
S5K(3-(PAO-amino)propionyl)-GLP-2(1-33);
S7K(3-(PAO-amino)propionyl)-GLP-2(1-33);
D8K(3-(PAO-amino)propionyl)-GLP-2(1-33);
E9K(3-(PAO-amino)propionyl)-GLP-2(1-33);
M10K(3-(PAO-amino)propionyl)-GLP-2(1-33);
N11K(3-(PAO-amino)propionyl)-GLP-2(1-33);
T12K(3-(PAO-amino)propionyl)-GLP-2(1-33);
I13K(3-(PAO-amino)propionyl)-GLP-2(1-33);
L14K(3-(PAO-amino)propionyl)-GLP-2(1-33);
D15K(3-(PAO-amino)propionyl)-GLP-2(1-33);
N16K(3-(PAO-amino)propionyl)-GLP-2(1-33);
L17K(3-(PAO-amino)propionyl)-GLP-2(1-33);
L17K((S)-4-carboxy-4-(PAO-amino)butanoyl)-GLP-2(1-33);
L17K((R)-4-carboxy-4-(PAO-amino)butanoyl)-GLP-2(1-33);
L17K(4-(PAO-amino)butanoyl)-GLP-2(1-33);
A18K(3-(PAO-amino)propionyl)-GLP-2(1-33);
D21K(3-(PAO-amino)propionyl)-GLP-2(1-33);
N24K(3-(PAO-amino)propionyl)-GLP-2(1-33);
Q28K(3-(PAO-amino)propionyl)-GLP-2(1-33);
S5K(3-(PAO-amino)propionyl)/K30R-GLP-2(1-33);
S7K(3-(PAO-amino)propionyl)/K30R-GLP-2(1-33);
D8K(3-(PAO-amino)propionyl)/K30R-GLP-2(1-33);
E9K(3-(PAO-amino)propionyl)/K30R-GLP-2(1-33);
M10K(3-(PAO-amino)propionyl)/K30R-GLP-2(1-33);
N11K(3-(PAO-amino)propionyl)/K30R-GLP-2(1-33);
T12K(3-(PAO-amino)propionyl)/K30R-GLP-2(1-33);
I13K(3-(PAO-amino)propionyl)/K30R-GLP-2(1-33);
L14K(3-(PAO-amino)propionyl)/K30R-GLP-2(1-33);
D15K(3-(PAO-amino)propionyl)/K30R-GLP-2(1-33);
N16K(3-(PAO-amino)propionyl)/K30R-GLP-2(1-33);
L17K(3-(PAO-amino)propionyl)/K30R-GLP-2(1-33);
L17K((S)-4-carboxy-4-(PAO-amino)butanoyl)/K30R-GLP-2(1-33);
L17K(4-(PAO-amino)butanoyl)/K30R-G LP-2(1-33);
A18K(3-(PAO-amino)propionyl)/K30R-GLP-2(1-33);
D21K(3-(PAO-amino)propionyl)/K30R-GLP-2(1-33);
N24K(3-(PAO-amino)propionyl)/K30R-GLP-2(1-33);
Q28K(3-(PAO-amino)propionyl)/K30R-GLP-2(1-33);
D3E/S5K(3-(PAO-amino)propionyl)/K30R/D33E-GLP-2(1-33);
D3E/S7K(3-(PAO-amino)propionyl)/K30R/D33E-GLP-2(1-33);
D3E/D8K(3-(PAO-amino)propionyl)/K30R/D33E-GLP-2(1-33);
D3E/E9K(3-(PAO-amino)propionyl)/K30R/D33E-GLP-2(1-33);
D3E/M10K(3-(PAO-amino)propionyl)/K30R/D33E-GLP-2(1-33);
D3E/N11K(3-(PAO-amino)propionyl)/K30R/D33E-GLP-2(1-33);
D3E/T12K(3-(PAO-amino)propionyl)/K30R/D33E-GLP-2(1-33);
D3E/I13K(3-(PAO-amino)propionyl)/K30R/D33E-GLP-2(1-33);
D3E/L14K(3-(PAO-amino)propionyl)/K30R/D33E-GLP-2(1-33);
D3E/D15K(3-(PAO-amino)propionyl)/K30R/D33E-GLP-2(1-33);
D3E/N16K(3-(PAO-amino)propionyl)/K30R/D33E-GLP-2(1-33);
D3E/L17K(3-(PAO-amino)propionyl)/K30R/D33E-GLP-2(1-33);
D3E/L17K((S)-4-carboxy-4-(PAO-amino)butanoyl)/K30R/D33E-GLP-2(1-33);
D3E/L17K(4-(PAO-amino)butanoyl)/K30R/D33E-GLP-2(1-33);
D3E/A18K(3-(PAO-amino)propionyl)/K30R/D33E-GLP-2(1-33);
D3E/D21K(3-(PAO-amino)propionyl)/K30R/D33E-GLP-2(1-33);
D3E/N24K(3-(PAO-amino)propionyl)/K30R/D33E-GLP-2(1-33);
D3E/Q28K(3-(PAO-amino)propionyl)/K30R/D33E-GLP-2(1-33);
S5K(N^{ε}-PAO)-GLP-2(1-33);
S7K(N^{ε}-PAO)-GLP-2(1-33);
D8K(N^{ε}-PAO)-GLP-2(1-33);
E9K(N^{ε}-PAO)-GLP-2(1-33);
M10K(N^{ε}-PAO)-GLP-2(1-33);
N11K(N^{ε}-PAO)-GLP-2(1-33);
T12K(N^{ε}-PAO)-GLP-2(1-33);
I13K(N^{ε}-PAO)-GLP-2(1-33);
L14K(N^{ε}-PAO)-GLP-2(1-33);
D15K(N^{ε}-PAO)-GLP-2(1-33);
N16K(N^{ε}-PAO)-GLP-2(1-33);
L17K(N^{ε}-PAO)-GLP-2(1-33);
A18K(N^{ε}-PAO)-GLP-2(1-33);
D21K(N^{ε}-PAO)-GLP-2(1-33);
N24K(N^{ε}-PAO)-GLP-2(1-33);
Q28K(N^{ε}-PAO)-GLP-2(1-33);
S5K(N^{ε}-PAO)/K30R-GLP-2(1-33);
S7K(N^{ε}-PAO)/K30R-GLP-2(1-33);
D8K(N^{ε}-PAO)/K30R-GLP-2(1-33);
E9K(N^{ε}-PAO)/K30R-GLP-2(1-33);
M10K(N^{ε}-PAO)/K30R-GLP-2(1-33);
N11K(N^{ε}-PAO)/K30R-GLP-2(1-33);
T12K(N^{ε}-PAO)/K30R-GLP-2(1-33);
I13K(N^{ε}-PAO)/K30R-GLP-2(1-33);
L14K(N^{ε}-PAO)/K30R-GLP-2(1-33);
D15K(N^{ε}-PAO)/K30R-GLP-2(1-33);
N16K(N^{ε}-PAO)/K30R-GLP-2(1-33);
L17K(N^{ε}-PAO)/K30R-GLP-2(1-33);
A18K(N^{ε}-PAO)propionyl)/K30R-GLP-2(1-33);
D21K(N^{ε}-PAO)/K30R-GLP-2(1-33);
N24K(N^{ε}-PAO)/K30R-GLP-2(1-33);
Q28K(N^{ε}-PAO)/K30R-GLP-2(1-33);
D3E/S5K(N^{ε}-PAO)/K30R/D33E-GLP-2(1-33);
D3E/S7K(3-(N^{ε}-PAO)/K30R/D33E-GLP-2(1-33);
D3E/D8K(N^{ε}-PAO)/K30R/D33E-GLP-2(1-33);
D3E/E9K(N^{ε}-PAO)/K30R/D33E-GLP-2(1-33);
D3E/M10K(N^{ε}-PAO)/K30R/D33E-GLP-2(1-33);
D3E/N11K(N^{ε}-PAO)/K30R/D33E-GLP-2(1-33);
D3E/T12K(N^{ε}-PAO)/K30R/D33E-GLP-2(1-33);
D3E/I13K(N^{ε}-PAO)/K30R/D33E-GLP-2(1-33);
D3E/L14K(N^{ε}-PAO)/K30R/D33E-GLP-2(1-33);
D3E/D15K(N^{ε}-PAO)/K30R/D33E-GLP-2(1-33);
D3E/N16K(N^{ε}-PAO)/K30R/D33E-GLP-2(1-33);
D3E/L17K(N^{ε}-PAO)/K30R/D33E-GLP-2(1-33);
D3E/A18K(N^{ε}-PAO)/K30R/D33E-GLP-2(1-33);
D3E/D21K(N^{ε}-PAO)/K30R/D33E-GLP-2(1-33);
D3E/N24K(N^{ε}-PAO)/K30R/D33E-GLP-2(1-33); and
D3E/Q28K(N^{ε}-PAO)/K30R/D33E-GLP-2(1-33).

17. A GLP-2 derivative according to claim 1, wherein the hydrophilic substituent comprises a sugar moiety

18. A GLP-2 derivative selected from
D3E/N16N(2-acetamido-2-deoxy-β-D-glucopyranosyl)/K30R-GLP-2(1-33) or
D3E/M10L/N11N(2-acetamido-2-deoxy-β-D-glucopyranosyl)-GLP-2(1-33).

19. A method for the treatment of intestinal failure or other condition leading to malabsorption of nutrients in the intestine comprising administering a therapeutically effective amount of a GLP-2 derivative according to claim 1 to a subject in need thereof.

20. A method for the treatment of a condition selected from inflammatory bowel syndrome, Crohn's disease, colitis, or osteoporosis, comprising administering a therapeutically effective amount of a GLP-2 derivative according to claim 1 to a subject in need thereof.


SEQUENCE LISTING (3 sequences)

SEQ ID NO: 1 - protein, 33 aa, Homo sapiens
HADGSFSDEMNTILDNLAARDFINWLIQTKITD

SEQ ID NO: 2 - protein, 33 aa, Artificial
  Synthetic
HXXGXFXXXXXXXXXXXXARXFIXWLIXTRITX

SEQ ID NO: 3 - protein, 33 aa, Artificial
  Synthetic
HXXGXFXXXXXXXXXXXXAXXFIXWLIXTXITX